(19) United States
(12) Patent Application Publication
(10) Pub. No.: US 2024/0229052 A1
(43) Pub. Date: Jul. 11, 2024

(54) Methods and Compositions for Improving Carbon Accumulation in Plants

(71) Applicants: David BECKER, La Verne, CA (US); Canisius University, Buffalo, NY (US)

(72) Inventors: David W. Becker, La Verne, CA (US); Robert J. Grebenok, East Amherst, CA (US)

(73) Assignee: Canisius University, Buffalo, NY (US)

(21) Appl. No.: 18/558,872

(22) PCT Filed: May 6, 2022
(86) PCT No.: PCT/US2022/028143

§ 371 Date: Nov. 3, 2023

Related U.S. Application Data
(60) Provisional application No. 63/188,622, filed on May 14, 2021.
(60) Provisional application No. 63/326,383, filed on Apr. 1, 2022.

(51) Int. Cl. C12N 15/82; A01G 7/06; C12N 9/04; C12P 7/18

(52) U.S. Cl. CPC C12N 15/8241; A01G 7/06; C12N 9/0006; C12P 7/18; C12Y 101/03006

(57) ABSTRACT

Disclosed herein are methods for decreasing plant generation time and increasing certain characteristics of plants including starch accumulation, root biomass, leaf biomass, stem biomass, and seed biomass. The description also includes methods for growing plants with less light increasing and/or shifting the latitude range of a plant so that the plant can be grown at higher latitudes. The improved plants can be used as food, or feedstock for making chemicals such as biofuels.

### BACKGROUND OF THE INVENTION

Plant biomass is composed predominantly of sugars, singly or combined by various linkages, and represents the greatest source of renewable hydrocarbon on earth. Unlike other renewable energy sources, biomass can be converted directly into liquid fuels. The two most common types of biofuels are ethanol (ethyl alcohol) and biodiesel. Ethanol is an alcohol, which can be produced by fermenting any biomass high in carbohydrates (starches, sugars, or celluloses). Once fermentable sugars have been obtained from the biomass material, these sugars can then be fermented to produce ethanol through a process similar to brewing beer. However, this enormous resource is under-utilized due to the fact sugars are locked in complex polymers, which are often referred to collectively as lignocellulose.

Carbohydrates constitute the most abundant organic compounds on earth. They are principally found in plants as complex glucose polymers either in the form of cellulose or starch. Cellulose, hemicellulose and glucans make up many structural components of the plant cell wall and woody tissues. These structural components are often complexed with other molecules such as proteins, fats and lignin. Starch is utilized by the plant as a principal short-term storage carbohydrate in leaves, and long-term storage carbohydrate in stems, modified stems such as tubers, roots and seeds, including grains. A biopolymer, starch consists of essentially pure linked glucose monomers. Starch is a desirable storage carbohydrate due to the fact that it is compositionally simple, stable, and can be readily broken down by the plant for energy. Comparatively, lignocellulosic material is composed of glucose and/or several different sugars complexed with lignin. Starch is readily hydrolysable to monomer sugars via effective and inexpensive starch-hydrolysing enzymes whereas lignocellulosic material is neither readily hydrolysable nor relatively inexpensive to process. Carbohydrates are also found in abundance in the form of the simple disaccharide sucrose. Sucrose may be found in crops such as sugarcane, sugarbeets, and sweet sorghum. Unlike sucrose, starch is stable and can be stored in dehydrated form for long periods of time.

### SUMMARY OF THE INVENTION

The description relates to methods for increasing starch accumulation and growth of transgenic plants by growing a plant that has been engineered to express cholesterol oxidase in the chloroplasts of the plant. Such transgenic plants grow faster than wild-type, and produce greater root biomass, seed biomass, stem biomass. These transgenic plants also have greater reproductive output and reach flowering in half the time of the wild-type plant. These increased characteristics of the transgenic plant are even greater when the plants are grown under light-limiting conditions. As a result of these improvements, the transgenic plants can be grown under lower light conditions (e.g., higher latitudes), can have multiple crop cycles in a single growing season, and produce greater crop yields per cycle. Root crops, seed/fruit crops, and grasses all can have increased output with the transgenic modification. The cholesterol oxidase can be a choM from Streptomyces sp. Strain A19249, found at GenBank Accession No. A19124. Cholesterol oxidase from a large number of other sources can also be used. A large number of bacterial species make cholesterol oxidase with the actinomycetes being a prolific group. Both pathogenic and nonpathogenic microorganisms make cholesterol oxidase including, for example, Mycobacterium, Brevibacterium, Streptomyces, Corynebacterium, Arthrobacter, Pseudomonas, Rhodococcus, Chromobacterium and Bacillus species. Any of the foregoing cholesterol oxidases can be engineered into the plants and algae, microalgae described herein.

Plants, algae and microalgae engineered to have cholesterol oxidase in the chloroplasts have about two-fold increased accumulation of starch. This increased starch is made primarily from CO₂ fixed from the air. Thus, the transgenic plants, algae and microalgae described herein utilize more CO₂ from the atmosphere than the wild-type plants, and the transgenic plants, algae and microalgae can be used to reduce CO₂ levels in the atmosphere. The increased starch in the plants, algae and microalgae can also be used for a variety of purposes including, for example, biofuel production, bioenergy, food production, green chemicals, and photovoltaic uses.

Plants and cells useful with the methods and compositions described herein include, for example, monocotyledonous or dicotyledonous plants, including, but not limited to, alfalfa, almonds, asparagus, avocado, banana, barley, bean, blackberry, brassicas, broccoli, cabbage, cannabis, canola, carrot, cauliflower, celery, cherry, chicory, citrus, coffee, cotton, cucumber, eucalyptus, hemp, lettuce, lentil, maize, mango, melon, oat, papaya, pea, peanut, pineapple, plum, potato (including sweet potatoes), pumpkin, radish, rapeseed, raspberry, rice, rye, sorghum, soybean, spinach, strawberry, sugar beet, sugarcane, sunflower, tobacco, tomato, turnip, wheat, zucchini, and other fruiting vegetables (e.g. tomatoes, pepper, chili, eggplant, cucumber, squash etc.), other bulb vegetables (e.g., garlic, onion, leek etc.), other pome fruit (e.g. apples, pears etc.), other stone fruit (e.g., peach, nectarine, apricot, pears, plums etc.), Arabidopsis species, woody plants such as coniferous and deciduous trees, an ornamental plant, a perennial grass, a forage crop, flowers, other vegetables, other fruits, other agricultural crops, herbs, grasses, or perennial plant parts (e.g., bulbs; tubers; roots; crowns; stems; stolons; tillers; shoots; cuttings, including un-rooted cuttings, rooted cuttings, and callus cuttings or callus-generated plantlets; apical meristems etc.) The term “plants” refers to all physical parts of a plant, including seeds, seedlings, saplings, roots, tubers, stems, stalks, foliage, flowers and fruits.

The algae and/or microorganism can include, for example, a photosynthetic microorganism from Actinochloris, Agmenellum, Amphora, Anabaena, Ankistrodesmus, Aphanizomenen, Arthrospyra, Asterochloris, Asteromonas, (Astephomene), Auxenochlorella, Basichlamys, Botrydiopsis, Botryococcus, Botryococcus, Botryokoryne, Boekelovia, Borodinella, Brachiomonas, Carteria, Cephaleuros, Chaetoceros, Chaetophora, Characiochloris, Characiosiphon, Chlainomonas, Chlamydomonas, Chlorella, Chlorochytrium, Chlorococcum, Chlorogonium, Chloroidium, Chlorokybus, Chloromonas, Chrysosphaera, Closteriopsis, Coccomyxa, Cricosphaera, Cryptomonas, Cyclotella, Desmotetra, Dictyochloris, Dictyochloropsis, Dunaliella, Ellipsoidon, Emiliania, Eremosphaera, Eudorina, Euglena, Fragilaria, Floydiella, Haematococcus, Hafniomonas, Heterochlorella, Gleocapsa, Gloeothamnion, Gongrosira, Gonium, Gungnir, Halosarcinochlamys, Hymenomonas, Isochrysis, Koliella, Lepocinclis, Lobocharacium, Lobochlamys, Lobomonas, Lobosphaera, Lobosphaeropsis, Marvania, Microglena, Monoraphidium, Myrmecia, Nannochloris, Nannochloropsis, Navicula, Nephrochloris, Nitzschia, Ochromonas, Oocystis, Oogamochlamys, Oscillatoria, Pabia, Pandorina, Parietochloris, Pascheria, Peridinium, Phacotus, Phaeodactylum, Phagus, Phormidium, Platydorina, Platymonas, Pleodorina, Pleurastrosarcina, Pleurochrysis, Polulichloris, Prasiola, Prasiolopsis, Prasiococcus, Prototheca, Pseudochlorella, Pseudocarteria, Pseudotrebouxia, Pteromonas, Pyrobotrys, Rhodomonas, Rhopatocystis, Rosenvingiella, Scenedesmus, Spirogyra, Stephanosphaera, Tetrabaena, Tetraedron, Tetraselmis, Tetraspora, Trebouxia, Trochisciopsis, Viridiella, Vitreochlamys, Volvox, Volvulina, Vulcanochloris, Watanabea, or Yamagishiella.

### BRIEF DESCRIPTION OF THE FIGURES

FIG. 1 shows starch accumulation in the leaves of wild-type and transgenic plants.

FIG. 2 shows the growth of transgenic plants and wild-type plants after about 7 and half weeks under low light conditions.

FIG. 3 shows a comparison of seed pod numbers per plant for transgenic versus wild-type plants grown under low light conditions.

FIG. 4 shows a comparison of seed pod numbers per plant for transgenic versus wild-type plants grown under low light conditions.

FIG. 5 shows a comparison of average seed weights per plant for transgenic versus wild-type plants grown under low light conditions.

FIG. 6 shows a comparison of average seed weights per plant for transgenic versus wild-type plants grown under low light conditions.

FIG. 7 shows a comparison of estimated seeds per plant for transgenic versus wild-type plants grown under low light conditions.

FIG. 8 shows a comparison of average root fresh weight per plant for transgenic versus wild-type plants grown under low light conditions.

FIG. 9 shows a comparison of average root dry-weight per plant for transgenic versus wild-type plants grown under low light conditions.

FIG. 10 shows a comparison of average leaf dry-weight per plant for transgenic versus wild-type plants grown under low light conditions.

FIG. 11 shows a comparison of average stem dry-weight per plant for transgenic versus wild-type plants grown under low light conditions.

FIG. 12 shows a comparison of total dry-weight per plant for transgenic versus wild-type plants grown under low light conditions.

FIG. 13 shows a comparison of average root fresh-weight per plant for transgenic versus wild-type plants grown under full sunlight conditions.

FIG. 14 shows a comparison of average root dry-weight per plant for transgenic versus wild-type plants grown under full sunlight conditions.

FIG. 15 shows a comparison of total leaf dry-weight per plant for transgenic versus wild-type plants grown under full sunlight conditions.

FIG. 16 shows a comparison of total stem dry-weight per plant for transgenic versus wild-type plants grown under full sunlight conditions.

FIG. 17 shows a comparison of total dry-weight per plant for transgenic versus wild-type plants grown under full sunlight conditions.

FIG. 18 shows a comparison of average number of flowers per plant for transgenic versus wild-type plants grown under full sunlight conditions.

FIG. 19 shows a comparison of average number of flowers per plant for transgenic versus wild-type plants grown under full sunlight conditions.

FIG. 20 shows a comparison of average seed and pod dry-weight per plant for transgenic versus wild-type plants grown under full sunlight conditions.

FIG. 21 shows a comparison of average seed and pod dry-weight per plant for transgenic versus wild-type plants grown under full sunlight conditions.

FIG. 22 shows a comparison of average number of seed pods per plant for transgenic versus wild-type plants grown under full sunlight conditions.

FIG. 23 shows a comparison of average number of seed pods per plant for transgenic versus wild-type plants grown under full sunlight conditions.

### DETAILED DESCRIPTION OF THE INVENTION

Before various embodiments of the present invention are further described, it is to be understood that this disclosure is not limited to its particular embodiments described, as such may, of course, vary. It is also to be understood that the terminology used herein is for the purposes of describing particular embodiments only, and is not intended to be limiting. It should be noted that references to “an” or “one” or “some” embodiment(s) in this disclosure are not necessarily to the same embodiment, and all such references mean at least one.

It is also to be understood that as used in the present disclosure and in the appended claims, the singular terms “a”, “an”, and “the” include plural referents unless context clearly indicates otherwise. Similarly, the word “or” is intended to include “and” (and vice versa) unless the context clearly indicates otherwise. Numerical limitations given with respect to concentrations or levels of a substance are intended to be approximate, unless the context clearly dictates otherwise. Thus, where a level is indicated to be at least (for example) 10 μg, it is intended that the level be understood to be at least approximately or about 10 μg.

### Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings.

As used herein, “biomass” refers to useful biological material, which material is to be collected and can be further processing to isolate or concentrate a product of interest. “Biomass” may comprise the fruit or parts of it or seeds, leaves, or stems or roots where these are the parts of the plant that are of particular interest for the industrial purpose. “Biomass”, as it refers to plant material, includes any structure or structures of a plant that contain or represent the product of interest.

As used herein, the term “cellular life cycle” refers to series of events involving the growth, replication, and division of a eukaryotic cell. Generally, it can be divided into five stages, known as G₀, in which the cell is quiescent, G₁ and G_{2,} in which the cell increases in size, S, in which the cell duplicates its DNA, and M, in which the cell undergoes mitosis and divides.

As used herein, “crop plant” refers to any plant that is cultivated for the purpose of producing plant material sought after by man or animal for either oral consumption, or for utilization in an industrial, pharmaceutical, or commercial process. Crop plants, include, but are not limited to maize, wheat, rice, barley, soybean, cotton, sorghum, beans in general, rape/canola, alfalfa, flax, sunflower, safflower, millet, rye, sugarcane, sugar beet, cocoa, tea, tropical sugar beet, Brassica, cotton, coffee, sweet potato, flax, peanut, clover; vegetables such as lettuce, tomato, cucurbits, cassava, potato, carrot, radish, pea, lentils, cabbage, cauliflower, broccoli, Brussels sprouts, peppers, and pineapple; tree fruits such as citrus, apples, pears, peaches, apricots, walnuts, avocado, banana, and coconut; and flowers such as orchids, carnations and roses. Other plants include perennial grasses, such as switchgrass, prairie grasses, Indiangrass, Big bluestem grass, miscanthus and the like. It is recognized that mixtures of plants may be used.

As used herein, the term “cytosol” refers to the portion of the cytoplasm not within membrane-bound sub-structures of the cell.

As used herein, the term “daughter cell” refers to cells that are formed by the division of a cell.

As used herein, the term “energy crop” refers to crops that may be favorable to use in a biomass conversion method in converting plant biomass to fuels or other chemicals. This group comprises but is not limited to sugarcane, sugarbeet, sorghum, switchgrass, miscanthus, wheat, rice, oat, barley and maize.

As used herein, the term “essential molecule” refers to a molecule needed by a cell for growth or survival.

As used herein, the term “genetically modified” refers to altering the genetic material of a cell so that a desired property or characteristic of the cell is changed. The term includes introduction of heterologous genetic material into the cell.

The term “harvest index” as defined herein refers to the ratio of biomass yield to the cumulative biomass at harvest. Two of the best energy crops today, cane and beets, in terms of harvest index, have limitations on storage stability, and have high moisture content at harvest. High moisture content has several disadvantages such as transportation costs for the harvest are higher since a greater proportion of the water needs to be moved with the crop. Storage stability is a significant issue, since there may be continued metabolism, or microbial contaminations that can lead to crop spoilage and sugar loss. Perishability of the crop has very different infrastructural implications for the movement, storage, and utilization of these types of agricultural products. An increase of the starch content would lead to a considerable increase of dry substance and storage stability.

As used herein, the term “heterologous” when used in reference to a nucleic acid or polypeptide refers to a nucleic acid or polypeptide not normally present in nature. Accordingly, a heterologous nucleic acid or polypeptide in reference to a host cell refers to a nucleic acid or polypeptide not naturally present in the given host cell. For example, a nucleic acid molecule containing a non-host nucleic acid encoding a polypeptide operably linked to a host nucleic acid comprising a promoter is considered to be a heterologous nucleic acid molecule. Conversely, a heterologous nucleic acid molecule can comprise an endogenous structural gene operably linked with a non-host (exogenous) promoter. Similarly, a peptide or polypeptide encoded by a non-host nucleic acid molecule, or an endogenous polypeptide fused to a non-host polypeptide is a heterologous peptide or polypeptide.

As used herein, the term “host cell” refers to a eukaryotic cell with which an artificial symbiont can associate.

The term “introducing” in the context of a polynucleotide, for example, a nucleotide construct of interest, is intended to mean presenting to the plant the polynucleotide in such a manner that the polynucleotide gains access to the interior of a cell of the plant.

As used herein, the term “parent cell” refers to a cell that divides to form two or more daughter cells.

As used herein, the term “phenotype” refers to the set of observable characteristics of an individual or cell resulting from the interaction of its genotype with the environment.

As used herein, the term “plant part” or “plant tissue” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like.

As used herein, the term “secrete” refers to the passing of molecules or signals from one side of a membrane to the other side.

As used herein the terms “stably introducing” or “stably introduced” are used interchangeably and in the context of a polynucleotide introduced into a plant mean the introduced polynucleotide is stably incorporated into the plant genome, and thus the plant is stably transformed with the polynucleotide.

As used herein the terms “stable transformation” or “stably transformed” is intended to mean that a polynucleotide, for example, a nucleotide construct described herein, introduced into a plant integrates into the genome of the plant and is capable of being inherited by the progeny thereof, more particularly, by the progeny of multiple successive generations.

As used herein, the term “transient transformation” in the context of a polynucleotide means that a polynucleotide is introduced into the plant and does not integrate into the genome of the plant.

### Increased Starch Accumulation

Starch is one of the most abundant polymers produced in nature and is synthesized as a storage carbohydrate throughout the plant kingdom. In storage organs it serves as a long-term carbon reserve, whereas in photosynthetically competent tissues it is transiently accumulated to provide both reduced carbon and energy during periods unfavorable for photosynthesis. Starch is a desirable storage carbohydrate because it is compositionally simple compared to cellulosic material, and it is very stable. Cellulosic material comprises several different sugars, complexed with lignin. Lignocellulose is extremely difficult to break down enzymatically. In contrast, starch is comprised of glucose and is readily hydrolysable to monomer sugars via effective and inexpensive starch-hydrolyzing enzymes. The accumulation of starch in green tissues and stems would provide a rich source for simple sugars in the plant biomass.

Starch comprises both linear (amylose) and branched (amylopectin) glucose polymers. Amylopectin from many, but not all plant sources contains phosphate-monoesters that are linked mainly to the C6 and C3 positions of glycosyl residues. The biochemical mechanism of starch phosphorylation has, however, only recently been elucidated. Transgenic potato plants (Lorberth et al (1998) Nat Biotechnol. 16(5):473-7, which is incorporated by reference in its entirety for all purposes) and the sex1 mutant of Arabidopsis (Yu et al. (2001) Plant Cell 13(8):1907-18, which is incorporated by reference in its entirety for all purposes) are deficient in a starch associated protein, which is herein referred to as R1, and they synthesize starch with decreased phosphate content. The purified recombinant R1-protein from potato is able to phosphorylate α-glucans (Ritte et al. (2002) Proc Natl Acad Sci USA 99(10):7166-71, which is incorporated by reference in its entirety for all purposes). It catalyzes a dikinase-type reaction, liberating the gamma-phosphate of ATP (resulting in the release of orthophosphate), but using the β-phosphate to phosphorylate glucosyl residues of the polyglucan. Because of this activity, the protein is considered a glucan, water dikinase (GWD) (Ritte et al. (2003) Planta 216(5):798-801, which is incorporated by reference in its entirety for all purposes).

Starch provide 80% of the world's calories. Starch serves as an important store of energy that is captured by plants using sunlight, water, carbon dioxide and soil nutrients. In photosynthesizing leaves, starch accumulates during the day and is remobilized at night to support continued respiration, sucrose export, and growth in the dark. The Calvin-Benson cycle in the chloroplast creates small chain carbohydrates that are used to make hexoses which get converted to starch and/or sucrose. The Calvin-Benson cycle evolved about 2 billion years ago and is the most abundant biochemical pathway on earth in terms of nitrogen investment, and plays the dominant role in the global carbon and oxygen cycles. Despite its evolutionary age, the Calvin-Benson cycle is unchanged from cyanobacteria to higher plants. This conservation of such an ancient pathway is remarkable.

Tobacco is a C3 photosynthesis plant, and so, is representative of other C3 photosynthetic plants including, for example, alfalfa (lucerne), barley, broad bean, cassava, Chlorella, cotton, cowpea, Eucalyptus, green beans, oats, rye, wheat, peanuts, potatoes, rice, spinach, soybean, sugar beets, sunflower, tomatoes, and most trees. Still other C3 plants include, for example, lawn grasses such as fescue and Kentucky bluegrass, evergreen trees and shrubs of the tropics, subtropics, and the Mediterranean, temperate evergreen conifers like the Scotch pine (Pinus sylvestris), deciduous trees and shrubs of the temperate regions, e.g. European beech (Fagus sylvatica), as well as weedy plants like the water hyacinth (Eichornia crassipes), lambsquarters (Chenopodium album), bindweed (Convolvolus arvensis), and wild oat (Avena fatua). In fact, 85% of all plants species use C3 photosynthesis.

C4 and Crassulacean Acid Metabolism (CAM) are variants of C3 photosynthesis that have evolved from the fundamental C3 type of photosynthesis. The Calvin-Benson Cycle is central to C3, C4 and CAM photosynthesis, with the differences occurring in how CO₂ is captured from the atmosphere, not in the chemical reduction, or fixation, of that atmospheric carbon. C4 and CAM plants capture atmospheric CO₂ in spatial and temporal separation, respectively, from the fixation of the acquired carbon. Regardless of method of carbon capture, the Calvin-Benson Cycle remains central to CO₂ assimilation in all photosynthetic plants. Furthermore, transitory starch synthesis occurs in direct association with the Calvin-Benson Cycle activity. In fact, starch synthesis is favored over sucrose synthesis under conditions of high rates of photosynthesis (Weise et al., 2011, J. Exp. Botany, vol. 62, pp. 3109-3118, which is incorporated by reference in its entirety for all purposes). Thus, C4 and CAM plants engineered with a cholesterol oxidase enzyme targeted to the chloroplasts where the Calvin-Benson Cycle resides would demonstrate higher rates of photosynthesis, and accordingly, higher levels of starch accumulation in the photosynthetic tissues. Highly productive C4 plants such as maize, sugarcane, sorghum, millet, switchgrass and Miscanthus would be strong candidates for such transformation. This would add value to the photosynthetic portions of these plants, whether harvested for starch directly, to be used in ethanolic fermentation for biofuels or industrial product synthesis, or harvested for biomass, to be used as silage or for biofuels.

All plants accumulate starch in plastids, called chloroplasts, in leaves or amyloplasts in storage tissues. Sucrose, made in the leaves, is transported to the storage organ, where it is imported into the cytosolic compartment of each cell. Fruits, seeds and tubers represent remarkable storage organs for energy (starch) and nutrients. In some plants such as rice, wheat, potato, cassava, and yam starch-storage tissue represents about 70% of the dry weight of the seed or tuber of which about 90% of the dry weight is starch.

Starch accumulation can be increased in transgenic plants by growing a plant that has been engineered to express cholesterol oxidase in the chloroplasts of the plant. The cholesterol oxidase enzyme is a bifunctional bacterial flavoprotein that catalyzes the oxidation and the isomerization of steroid substrates containing a C3 hydroxyl. ChOx (cholesterol oxidase) catalyzes the following steps:

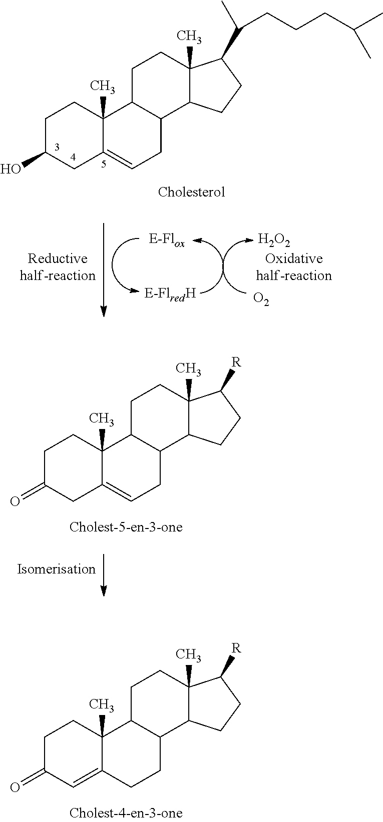

Members of the Cholesterol oxidase family of enzymes produce cholest-4-en-3-one steroids from cholesterol and an equimolar amount of hydrogen peroxide per reaction. The family of Cholesterol oxidase enzymes is divided into 2 categories based on the association of the FAD cofactor with the enzyme. Type I Cholesterol oxidases have an FAD non-covalently linked to the enzyme, while in the type II enzymes the FAD is covalently linked to the active site of the protein. Those Cholesterol oxidase enzymes maintaining the non-covalent association with FAD belong to the glucose-methanol-choline oxidoreductase flavoenzyme group, whereas those members of the Cholesterol oxidase family with the covalent linkage of FAD belong to the vanillyl-alcohol oxidase group of oxidoreductases. The 3D structures of the two types of Cholesterol oxidase enzymes show completely different tertiary organization but catalyze the same reaction. The enzymes belonging to the Type I Cholesterol oxidase subfamily include those enzymes isolated from the organisms, which include the Streptomyces sp., Rhodococcus equi, and Nostoc sp., while the Cholesterol oxidase enzymes belonging to the Type II subfamily include the enzymes isolated from the organisms Brevibacterium sterolicum, Burkholderia cepacia and Chromobacterium sp.

Cholesterol oxidase is produced by a large number of bacterial species, and the actinomycetes being most prolific group. Cholesterol oxidases are produced by microorganisms of both pathogenic and nonpathogenic nature such as Mycobacterium, Brevibacterium, Streptomyces, Corynebacterium, Arthrobacter, Pseudomonas, Rhodococcus, Chromobacterium and Bacillus species.

The cholesterol oxidase can be a choM from Streptomyces sp. Strain A19249, found at GenBank Accession No. A19124. Other cholesterol oxidase genes that can be used include, for example, Streptomyces sp. (choA) GenBank Acc. No. M31939, Streptomyces virginiae (choL) GenBank Acc. No. EU013931, Brevibacterium sp. (choB) GenBank Acc. DQ34780, Brevibacterium sterolicum (choB) GenBank Acc. No. D00712, Acineotobacter baumanii (choA) GenBank Acc. No. MK575469, Synthetic construct clone 15 (choA) GenBank Acc. No. MH892608, Synthetic construct GenBank Acc. No. MH794365, Arthrobacter sp. (choA) GenBank Acc. No. KY305682, Pseudomonas aeruginosa (choP) GenBank Acc. No. AB920752, Chromobacterium sp. DS-1 GenBank Acc. No. AB456533, Streptomyces sp. 769 (choA) GenBank Acc. No. KF290994, Mycobacterium sp. (choD) GenBank Acc. No. GU222349, Streptomyces sp. (choM) GenBank Acc. No. U13981—our construct, Nocardioides simplex (COX) GenBank Acc. No. AF247810, Arthrobacter sp. (choF) GenBank Acc. No. AY963570, Rhodococcus sp. GenBank Acc. No. DQ629027, Burkholderia cepacia ST-200 (choS) GenBank Acc. No. AB051408, Burkholderia cepacia ZWS15 GenBank Acc. No. MK757498, Synthetic construct (choA) GenBank Acc. No. MN013851, Gaeumannomyces tritici mRNA GenBank Acc. No. XM_009224693, Pseudomonas aeruginosa GenBank Acc. No. KU315227, Exophiala dematidis GenBank Acc. No. XM_009162790, Rhodococcus equi WGC1 (choE) GenBank Acc. No. KF670817, Nostoc sp. GenBank Acc. No. KC539822, Mycobacterium neoaurum NwIB-01 (choM1) Acc. No. JQ303323, Mycobacterium neoaurum (choM) GenBank Acc. No. JQ303324, Gordonia cholesterolivorans (cho2) GenBank Acc. No. GU320251, Gordonia cholesterolivorans (cho1) GenBank Acc. No. GU320250, Streptomyces griseu (choG) GenBank Acc. No. DQ135989, Rodococcus equi (choE) GenBank Acc. No. AJ242746.

Cholesterol oxidase can be encoded as a precursor that contains a special “zip code,” a targeting sequence specific to the intended final destination of a given protein. The “zip code” is located at the precursor N-terminus, appropriately called a transit peptide (TP). Transit peptides direct translocation of precursor proteins across the double membranes of plastids via the translocon at the TOC/TIC complex in a process described as the general import pathway. After the precursor is translocated into the stroma, the transit peptide is readily cleaved allowing the mature domain to fold into its native conformation or to be further targeted to the thylakoid.

The cholesterol oxidase protein and gene can be engineered to be operably linked to a transit peptide such as, for example, the transit peptide from the rubisco small subunit (Arabidopsis thaliana). Transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; Shah et al. (1986) Science 233:478-481, U.S. Pat. Nos. 5,510,471 and 5,633,448, all of which are incorporated by reference in their entirety for all purposes. Transit peptides also include those for chloroplasts or other plastids from plant genes whose gene product is targeted to the plastids, such as the chloroplast transit peptides described by Van Den Broeck et al. Nature, vol. 313, Jan. 1985, p. 358-363, the optimized transit peptide described by U.S. Pat. No. 5,635,618, the transit peptide of ferredoxin-NADP+oxidoreductase from spinach (Oelmuller et al., 1993, Mol. Gen. Genet., vol. 237, pp. 261-272), the transit peptide described in Wong et al. Plant Molec. Biol., vol. 20, pp. 81-93 (1992), or the targeting peptides in published PCT patent application WO 00/26371, all of which are incorporated by reference in their entirety for all purposes. Transit peptides are also described in Plant Molecular Biology (1998), devoted in large part to the transport of proteins into the various compartments of the plant cell (Sorting of proteins to vacuoles in plant cells pp 127-144; the nuclear pore complex pp 145-162; protein translocation into and across the chloroplast envelope membranes pp 91-207; multiple pathways for the targeting of thylakoid proteins in chloroplasts pp 209-221; mitochondrial protein import in plants pp 311-338), all of which are incorporated by reference in their entirety for all purposes.

Other plastids in the cell can also be modified with cholesterol oxidase by the transit peptide constructs. Cholesterol oxidase and other enzymes could be used to change the color of pigments in flowers and other plant parts.

The gene encoding cholesterol oxidase can also be engineered into the genome of the chloroplast.

The expression of cholesterol oxidase in chloroplasts changes the composition of the steroids in the chloroplast membranes. Cholesterol oxidase localization to the chloroplast increases light utilization by about two-fold and this increased energy is converted into two-fold as much starch accumulation in the plant. This also should translate into two-fold as much CO₂ absorbed by these plants (double the carbon fixation of CO₂). Thus, these plants have double the carbon available for energy and synthesis of compounds/products. Sterols-steroids can affect membrane fluidity, and can interact directly/indirectly with the electron chain complex components. Sterols-steroids can affect the efficiency of energy transfer in the chlorophyll antenna complexes—this is doubled in the presence of cholesterol oxidase. This effect can arise from increased ordering of the chlorophyll units and/or modification of the chlorophyll environment that increases energy transfer. This increase in energy transfer results in commensurate increases in photosynthetic electron transport.

Cholesterol oxidase expression in the chloroplasts results in a two-fold increase (100%) in starch production in the plant—the excess energy from the photosynthetic electron transport is translated into increased starch production (starch is a form of stored energy) in the plant cells.

### Improved Properties of Transgenic Plants

The transgenic modifications and plants described herein also have other improved properties. Root biomass, seed biomass, stem biomass, and leaf biomass were all increased in the transgenic plants. In addition, the transgenic plants had increased reproductive output, and the time to flowering was reduced by 50%. The transgenic plants have very significant growth advantages grown in full sunlight conditions, and that these advantages are substantially greater when plants are grown under light-limiting conditions. The increased photosynthetic electron transport capacity and light use efficiency of the transgenic chloroplasts confers these growth enhancements on the transgenic plants through increased rates of photosynthesis. These improvements can be achieved in any plants that are transgenically modified as described herein as chloroplasts in all plants can have increased performance with the transgenic modification described herein.

The increased performance of the transgenic plants can allow for longer growing seasons as the transgenic plants can grow with shorter days of sunlight due to the increased efficiency of the chloroplasts. Similarly, the latitudes at which the transgenic plants may be grown is also expanded by the transgenic modification as the increased efficiency of the chloroplasts can allow the transgenic plants to grow with reduced sunlight intensity. The more rapid development of the transgenic plants can also allow multiple crops to be grown and harvested in one growing season.

The increased properties seen in the transgenic plants improve yields of roots, stems, leaves and seeds/fruit. Thus, many root crops, seed/fruit crops, grasses, and other crops can have improved production and efficiencies with this transgenic modification.

### Plant Transformation

Expression cassettes carrying genes of interest can be introduced into plant cells in a number of art-recognized ways. Where more than one polynucleotide is to be introduced, these polynucleotides can be assembled as part of a single nucleotide construct, or as separate nucleotide constructs, and can be located on the same or different transformation vectors. Accordingly, these polynucleotides can be introduced into the host cell of interest in a single transformation event, in separate transformation events, or, for example, in plants, as part of a breeding protocol. The methods of the invention do not depend on a particular method for introducing one or more polynucleotides into a plant, only that the polynucleotide(s) gains access to the interior of at least one cell of the plant. Methods for introducing polynucleotides into plants are known in the art including, but not limited to, transient transformation methods, stable transformation methods, and virus-mediated methods.

Numerous transformation vectors are available for plant transformation, and the genes encoding cholesterol oxidase can be used in conjunction with any such vectors. The selection of vector will depend upon the preferred transformation technique and the target species for transformation. For certain target species, different antibiotic or herbicide selection markers may be preferred. Selection markers used routinely in transformation include the npt11 gene, which confers resistance to kanamycin and related antibiotics (Messing & Vierra. Gene 19: 259-268 (1982); Bevan et al., Nature 304:184-187 (1983), both of which are incorporated by reference in their entirety for all purposes), the bar gene, which confers resistance to the herbicide phosphinothricin (White et al., Nucl. Acids Res 18: 1062 (1990), Spencer et al. Theor. Appl. Genet 79: 625-631 (1990), both of which are incorporated by reference in their entirety for all purposes), the hph gene, which confers resistance to the antibiotic hygromycin (Blochinger & Diggelmann, Mol Cell Biol 4: 2929-2931, which is incorporated by reference in its entirety for all purposes), and the dhfr gene, which confers resistance to methotrexate (Bourouis et al., EMBO J. 2(7): 1099-1104 (1983), which is incorporated by reference in its entirety for all purposes), the EPSPS gene, which confers resistance to glyphosate (U.S. Pat. Nos. 4,940,935 and 5,188,642), both of which are incorporated by reference in their entirety for all purposes), and the mannose-6-phosphate isomerase gene, which provides the ability to metabolize mannose (U.S. Pat. Nos. 5,767,378 and 5,994,629), both of which are incorporated by reference in their entirety for all purposes).

Methods for regeneration of plants are also known. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Many vectors are available for transformation using Agrobacterium tumefaciens. These typically carry at least one T-DNA border sequence and include vectors such as pBIN19 (Sevan, Nucl. Acids Res. (1984), which is incorporated by reference in its entirety for all purposes). For the construction of vectors useful in Agrobacterium transformation, see, for example, US Patent Application Publication No. 2006/0260011, which is incorporated by reference in its entirety for all purposes.

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. For the construction of such vectors, see, for example, US Application No. 20060260011, which is incorporated by reference in its entirety for all purposes.

For expression of a nucleotide sequence of the present invention in plant plastids, plastid transformation vector pPH143 (WO 97/32011, which is incorporated by reference in its entirety for all purposes) is used. The nucleotide sequence is inserted into pPH143 thereby replacing the PROTOX coding sequence. This vector is then used for plastid transformation and selection of transformants for spectinomycin resistance. Alternatively, the nucleotide sequence is inserted in pPH143 so that it replaces the aadH gene. In this case, transformants are selected for resistance to PROTOX inhibitors.

Transformation techniques for dicotyledonous plants are well known and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J. 3: 2717-2722 (1984), Potrykus et al., Mol. Gen. Genet. 199: 169-177 (1985), Reich et al., Biotechnology 4: 1001-1004 (1986), and Klein et al., Nature 327: 70-73 (1987), all of which are incorporated by reference in their entirety for all purposes. In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain which may depend on the complement of vir genes carried by the host Agrobacterium strain either on a co-resident Ti plasmid or chromosomally (e.g. strain CIB542 for pCIB200 and pCIB2001 (Uknes et al. Plant Cell 5: 159-169 (1993), which is incorporated by reference in its entirety for all purposes). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen &. Willmitzer, Nucl. Acids Res. 16: 9877 (1988), which is incorporated by reference in its entirety for all purposes).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050, 5,036,006, and 5,100,792, all of which are incorporated by reference in their entirety for all purposes. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

Transformation of most monocotyledon species is also routine. Preferred techniques include direct gene transfer into protoplasts using PEG or electroporation techniques, and particle bombardment into callus tissue. Transformations can be undertaken with a single DNA species or multiple DNA species (i.e. co-transformation) and both of these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable.

Techniques for the preparation of callus and protoplasts from an elite inbred line of maize, transformation of protoplasts using PEG or electroporation, and the regeneration of maize plants from transformed protoplasts are described in Patent Applications EP 0 292 435, EP 0 392 225, and WO 93/07278, all of which are incorporated by reference in their entirety for all purposes. Published techniques for transformation of A188-derived maize line using particle bombardment is also known, Gordon-Kamm et al. (Plant Cell 2: 603-618 (1990)) and Fromm et al. (Biotechnology 8: 833-839 (1990)) both of which are incorporated by reference in their entirety for all purposes. Furthermore, techniques for the transformation of elite inbred lines of maize by particle bombardment are known, WO 93/07278 and Koziel et al. (Biotechnology 11: 194-200 (1993)) both of which are incorporated by reference in their entirety for all purposes. This technique utilizes immature maize embryos of 1.5-2.5 mm length excised from a maize ear 14-15 days after pollination and a PDS-1000He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al. Plant Cell Rep 7: 379-384 (1988); Shimamoto et al. Nature 338: 274-277 (1989); Datta et al. Biotechnology 8: 736-740 (1990) all of which are incorporated by reference in their entirety for all purposes). Both types are also routinely transformable using particle bombardment (Christou et al. Biotechnology 9: 957-962 (1991) which is incorporated by reference in its entirety for all purposes). Furthermore, techniques for the transformation of rice via electroporation are known, e.g., WO 93/21335 which is incorporated by reference in its entirety for all purposes.

Techniques for the generation, transformation and regeneration of Pooideae protoplasts are also known, see e.g., Patent Application EP 0 332 581 which is incorporated by reference in its entirety for all purposes. These techniques allow the transformation of Dactylis and wheat. Furthermore, wheat transformation using particle bombardment into cells of type C long-term regenerable callus has been described by Vasil et al. (Biotechnology 10: 667-674 (1992) which is incorporated by reference in its entirety for all purposes), and also using particle bombardment of immature embryos and immature embryo-derived callus as described by Vasil et al. (Biotechnologyl 11:1553-1558 (1993)) and Weeks et al. (Plant Physiol. 102: 1077-1084 (1993)) both of which are incorporated by reference in their entirety for all purposes. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashiga & Skoog, Physiologia Plantarum 15: 473-497 (1962), which is incorporated by reference in its entirety for all purposes) and 3 mg/l 2,4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 hours and are then bombarded. Twenty embryos per target plate is typical, although not critical. An appropriate gene-carrying plasmid (such as pCIB3064 or pSOG35) is precipitated onto micrometer size gold particles using standard procedures. Each plate of embryos is shot with the DuPont BIOLISTICS® helium device using a burst pressure of about 1000 psi using a standard 80 mesh screen. After bombardment, the embryos are placed back into the dark to recover for about 24 hours (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Transformation of monocotyledons using Agrobacterium has also been described. See, WO 94/00977 and U.S. Pat. No. 5,591,616, both of which are incorporated by reference in their entirety for all purposes. See also, Negrotto et al., Plant Cell Reports 19: 798-803 (2000), which is incorporated by reference in its entirety for all purposes.

For example, rice (Oryza sativa) can be used for generating transgenic plants. Various rice cultivars can be used (Hiei et al., 1994, Plant Journal 6:271-282; Dong et al., 1996, Molecular Breeding 2:267-276; Hiei et al., 1997, Plant Molecular Biology, 35:205-218, all of which are incorporated by reference in their entirety for all purposes). Also, the various media constituents described below may be either varied in quantity or substituted. Embryogenic responses are initiated and/or cultures are established from mature embryos by culturing on MS-CIM medium (MS basal salts, 4.3 g/liter; B5 vitamins (200×), 5 ml/liter; Sucrose, 30 g/liter; proline, 500 mg/liter; glutamine, 500 mg/liter; casein hydrolysate, 300 mg/liter; 2,4-D (1 mg/ml), 2 ml/liter; adjust pH to 5.8 with 1 N KOH; Phytagel, 3 g/liter). Either mature embryos at the initial stages of culture response or established culture lines are inoculated and co-cultivated with the Agrobacterium tumefaciens strain LBA4404 (Agrobacterium) containing the desired vector construction. Agrobacterium is cultured from glycerol stocks on solid YPC medium (100 mg/L spectinomycin and any other appropriate antibiotic) for about two days at 28° C. Agrobacterium is re-suspended in liquid MS-CIM medium. The Agrobacterium culture is diluted to an OD600 of 0.2-0.3 and acetosyringone is added to a final concentration of 200 □M. Acetosyringone is added before mixing the solution with the rice cultures to induce Agrobacterium for DNA transfer to the plant cells. For inoculation, the plant cultures are immersed in the bacterial suspension. The liquid bacterial suspension is removed and the inoculated cultures are placed on co-cultivation medium and incubated at 22° C. for two days. The cultures are then transferred to MS-CIM medium with Ticarcillin (400 mg/liter) to inhibit the growth of Agrobacterium. For constructs utilizing the PMI selectable marker gene (Reed et al., In Vitro Cell. Dev. Biol.-Plant 37:127-132), cultures are transferred to selection medium containing Mannose as a carbohydrate source (MS with 2% Mannose, 300 mg/liter Ticarcillin) after 7 days, and cultured for 3-4 weeks in the dark. Resistant colonies are then transferred to regeneration induction medium (MS with no 2,4-D, 0.5 mg/liter IAA, 1 mg/liter zeatin, 200 mg/liter timentin 2% Mannose and 3% Sorbitol) and grown in the dark for 14 days. Proliferating colonies are then transferred to another round of regeneration induction media and moved to the light growth room. Regenerated shoots are transferred to GA7 containers with GA7-1 medium (MS with no hormones and 2% Sorbitol) for 2 weeks and then moved to the greenhouse when they are large enough and have adequate roots. Plants are transplanted to soil in the greenhouse (To generation) grown to maturity, and the T₁ seed is harvested.

The plants obtained via transformation with a nucleic acid sequence described herein can be any of a wide variety of plant species, including those of monocots and dicots; however, the plants used in the method of the invention are preferably selected from the list of agronomically important target crops set forth supra. The expression of a gene described herein in combination with other characteristics important for production and quality can be incorporated into plant lines through breeding. Breeding approaches and techniques are known in the art. See, for example, Welsh J. R., Fundamentals of Plant Genetics and Breeding, John Wiley & Sons, NY (1981); Crop Breeding, Wood D. R. (Ed.) American Society of Agronomy Madison, Wis. (1983); Mayo O., The Theory of Plant Breeding, Second Edition, Clarendon Press, Oxford (1987); Singh, D. P., Breeding for Resistance to Diseases and insect Pests, Springer-Verlag, NY (1986); and Wricke and Weber, Quantitative Genetics and Selection Plant Breeding, Walter de Gruyter and Co., Berlin (1986), all of which are incorporated by reference in their entirety for all purposes.

For the transformation of plastids, seeds of Nicotiana tabacum c.v. Xanthi are germinated seven per plate in a 1″ circular array on T agar medium and bombarded 12-14 days after sowing with 1 um tungsten particles (M10, Biorad, Hercules, Calif.) coated with DNA from plasmids pPH143 and pPH145 essentially as described (Svab, Z. and Maliga, P. (1993) PNAS 90, 913-917, which is incorporated by reference in its entirety for all purposes). Bombarded seedlings are incubated on T medium for two days after which leaves are excised and placed abaxial side up in bright light (350-500 umol photons/m.sup.2/s) on plates of RMOP medium (Svab. Z., Hajdukiewicz, P. and Maliga, P. (1990) PNAS 87, 8526-8530, which is incorporated by reference in its entirety for all purposes) containing 500 ug/ml spectinomycin dihydrochloride (Sigma, St. Louis, Mo.). Resistant shoots appearing underneath, the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook et al., (1989) Molecular Cloning: A Laboratory Manual, Cold Spring Harbor Laboratory, Cold Spring Harbor). BamHI/EcoRI-digested total cellular DNA (Mettler, I. J. (1987) Plant Mol Biol Reporter 5, 346349) is separated on 1% Tris-borate (TBE) agarose gels, transferred to nylon membranes (Amersham) and probed with ³²P-labeled random primed DNA sequences corresponding to a 0.7 kb BamHI/HindIII DNA fragment from pC8 containing a portion of the rps 7/12 plastid targeting sequence. Homoplasmic shoots are rooted aseptically on spectinomycin-containing MS/IBA medium (McBride, K. E. et al. (1994) PNAS 91, 7301-7305, which is incorporated by reference in its entirety for all purposes) and transferred to the greenhouse.

The genetic properties engineered into the transgenic seeds and plants described above are passed on by sexual reproduction or vegetative growth and can thus be maintained and propagated in progeny plants. Generally, maintenance and propagation make use of known agricultural methods developed to fit specific purposes such as tilling, sowing or harvesting.

Use of the advantageous genetic properties of the transgenic plants and seeds described herein can further be made in plant breeding. Depending on the desired properties, different breeding measures are taken. The relevant techniques are well known and include but are not limited to hybridization, inbreeding, backcross breeding, multi-line breeding, variety blend, interspecific hybridization, aneuploid techniques, etc. Thus, the transgenic seeds and plants can be used for the breeding of improved plant lines that, for example, increase the effectiveness of conventional methods such as herbicide or pesticide treatment or allow one to dispense with said methods due to their modified, genetic properties.

### Plants and Plant Cells

The plant or plant cells can be of monocotyledonous or dicotyledonous plants, including, but not limited to, alfalfa, almonds, asparagus, avocado, banana, barley, bean, blackberry, brassicas, broccoli, cabbage, canola, carrot, cauliflower, celery, cherry, chicory, citrus, coffee, cotton, cucumber, eucalyptus, hemp, lettuce, lentil, maize, mango, melon, oat, papaya, pea, peanut, pineapple, plum, potato (including sweet potatoes), pumpkin, radish, rapeseed, raspberry, rice, rye, sorghum, soybean, spinach, strawberry, sugar beet, sugarcane, sunflower, tobacco, tomato, turnip, wheat, zucchini, and other fruiting vegetables (e.g. tomatoes, pepper, chili, eggplant, cucumber, squash etc.), other bulb vegetables (e.g., garlic, onion, leek etc.), other pome fruit (e.g. apples, pears etc.), other stone fruit (e.g., peach, nectarine, apricot, pears, plums etc.), Arabidopsis, woody plants such as coniferous and deciduous trees, an ornamental plant, a perennial grass, a forage crop, flowers, other vegetables, other fruits, other agricultural crops, herbs, grass, or perennial plant parts (e.g., bulbs; tubers; roots; crowns; stems; stolons; tillers; shoots; cuttings, including un-rooted cuttings, rooted cuttings, and callus cuttings or callus-generated plantlets; apical meristems etc.). The term “plants” refers to all physical parts of a plant, including seeds, seedlings, saplings, roots, tubers, stems, stalks, foliage and fruits.

The plant or plant cells can be plants that use C3 photosynthesis including, for example, alfalfa (lucerne), barley, broad bean, cassava, Chlorella, cotton, cowpea, Eucalyptus, green beans, oats, rye, wheat, peanuts, potatoes, rice, spinach, soybean, sugar beets, sunflower, tomatoes, and most trees. Still other C3 plants include, for example, lawn grasses such as fescue and Kentucky bluegrass, evergreen trees and shrubs of the tropics, subtropics, and the Mediterranean, temperate evergreen conifers like the Scotch pine (Pinus sylvestris), deciduous trees and shrubs of the temperate regions, e.g. European beech (Fagus sylvatica), as well as weedy plants like the water hyacinth (Eichornia crassipes), lambsquarters (Chenopodium album), bindweed (Convolvolus arvensis), and wild oat (Avena fatua).

Plants and plant cells can also include algae, for example, algae of the genera Chlorella, Chlamydomonas, Scenedesmus, Isochrysis, Dunaliella, Tetraselmis, Nannochloropsis, or Prototheca.

The plant or plant cell can be from an indeterminate plant. These varieties grow vegetatively for indefinite periods in temperate regions. An indeterminate plant can be engineered to accumulate starch in green tissues and can be grown until the first frost. At that time, the plant could be allowed to desiccate, harvested dry, and used for food, livestock feed, or in biomass conversion processes.

The plant or plant cell can be from a photoperiod sensitive plant. One example of a photoperiod sensitive plant would be a tropical maize variety which when grown in the Midwest (or comparable long day summer climates) the plant will grow tall and generate little or no ears of maize. This in turn allows the tropical maize variety to have a large amount of green tissue biomass and accumulate sugars mainly in the form of sucrose in the plant's stalks and leaves. The current invention would convert these sucrose-storing photoperiod sensitive plants into starch-storing plants. Thus, increasing the value of the photoperiod sensitive plant and its' biomass storage stability.

The plant or plant cell includes algae and/or microalgae which can be, for example, a photosynthetic, or non-photosynthetic, microorganism from Actinochloris, Agmenellum, Amphora, Anabaena, Ankistrodesmus, Aphanizomenen, Arthrospyra, Asterochloris, Asteromonas, (Astephomene), Auxenochlorella, Basichlamys, Botrydiopsis, Botryococcus, Botryococcus, Botryokoryne, Boekelovia, Borodinella, Brachiomonas, Carteria, Cephaleuros, Chaetoceros, Chaetophora, Characiochloris, Characiosiphon, Chlainomonas, Chlamydomonas, Chlorella, Chlorochytrium, Chlorococcum, Chlorogonium, Chloroidium, Chlorokybus, Chloromonas, Chrysosphaera, Closteriopsis, Coccomyxa, Cricosphaera, Cryptomonas, Cyclotella, Desmotetra, Dictyochloris, Dictyochloropsis, Dunaliella, Ellipsoidon, Emiliania, Eremosphaera, Eudorina, Euglena, Fragilaria, Floydiella, Haematococcus, Hafniomonas, Heterochlorella, Gleocapsa, Gloeothamnion, Gongrosira, Gonium, Gungnir, Halosarcinochlamys, Hymenomonas, Isochrysis, Koliella, Lepocinclis, Lobocharacium, Lobochlamys, Lobomonas, Lobosphaera, Lobosphaeropsis, Marvania, Microglena, N Monoraphidium, Myrmecia, Nannochloris, Nannochloropsis, Navicula, Nephrochloris, Nitzschia, Ochromonas, Oocystis, Oogamochlamys, Oscillatoria, Pabia, Pandorina, Parietochloris, Pascheria, Peridinium, Phacotus, Phaeodactylum, Phagus, Phormidium, Platydorina, Platymonas, Pleodorina, Pleurastrosarcina, Pleurochrysis, Polulichloris, Prasiola, Prasiolopsis, Prasiococcus, Prototheca, Pseudochlorella, Pseudocarteria, Pseudotrebouxia, Pteromonas, Pyrobotrys, Rhodomonas, Rhopatocystis, Rosenvingiella, Scenedesmus, Spirogyra, Stephanosphaera, Tetrabaena, Tetraedron, Tetraselmis, Tetraspora, Trebouxia, Trochisciopsis, Viridiella, Vitreochlamys, Volvox, Volvulina, Vulcanochloris, Watanabea, or Yamagishiella.

### Methods of Use of Transgenic Plants

The transgenic plants with heterologous cholesterol oxidase in their chloroplasts can be used for many applications. Exemplary applications include biofuel production, bioenergy, food production, green chemicals, photovoltaic uses, etc.

The increased starch made in transgenic plants can be used as precursor/carbon sources for the making of biofuels (e.g., ethanol), industrial chemicals (e.g., butanediol), and other chemicals. In one application cholesterol oxidase is engineered into plants that grow quickly and have large leaves (e.g., plants referred to as weeds). The cholesterol oxidase can also be engineered into algae and microalgae for biofuel (and other chemical) production, industrial chemical production, and the production of other chemicals. When the plants, algae, or microalgae are engineered with cholesterol oxidase, the plants, algae or microalgae increases production of plant biomass (e.g., starch) which can be utilized to make biofuels, industrial chemicals, and other chemicals.

Cholesterol oxidase can be engineered into microalgae to increase energy production for making chemical products in microalgae such as those described in, for example, Cinar et al., Bioplastic production from microalgae: a review, 2020, Int. J. Environ. Res. Public Health 17:3842 (doi: 10.3390/ijerph17113842), Coppola et al., Bioplastic from renewable biomass: a facile solution for a greener environment, 2021, Earth Systems and Environment, doi.org/10.1007/s41748-021-00208-7, all of which are incorporated by reference in their entirety for all purposes. Further the microalgae described above, or other microorganisms engineered for making chemicals can use biomass enriched for starch from plants engineered with cholesterol oxidase. Such engineered microorganisms include the above microalgae and those described, for example, in Muniyandi et al., Perspectives of bioplastics—review, 2020, Int'l J. Scientific & Technol. Res. 9:374-381, Hong et al., Review of bioplastics as food packaging materials, 2021, AIMS Material Sciences 8:166-184, Temesgen et al., Review of spinning of biopolymer fibers from starch, 2021, Polymers 13:1121 (doi.org/10.3390/polym13071121), Venkatachalam et al., Bioplastic world: a review, 2020, J. Adv. Sci. Res. 11:43-53, Shah et al., Bioplastic for future: a review of then and now, 2021, World J. Adv. Res. Rev. 9:56-67, Hwang et al., Sustainable bioplastics: recent progress in the production of bio-building blocks for the bio-based next-generation polymer PEF, 2020, Chem. Engineer. J. 390:124636, all of which are incorporated by reference in their entirety for all purposes.

The chloroplasts in the transgenic plants (chloroplasts with cholesterol oxidase) can utilize harvested light about two-fold more efficiently than wild-type chloroplasts. The harvested light energy is transformed into cellular chemical energy and can be used to drive energy-requiring cellular processes, including chemically reducing CO₂ to carbohydrate. The chloroplasts from the transgenic plants can be used to increase light use efficiency in a variety of applications.

The transgenic plants can increase starch accumulation in the plants by fixing more CO₂ from the air. Thus, the transgenic plants can be used to remove excess CO₂ from the atmosphere. As these transgenic plants double the CO₂ fixed into starch in the plant, these transgenic plants will absorb more CO₂ from the atmosphere. Such carbon capture methods using the transgenic plants could address the excess CO₂ in the atmosphere. In addition, biofuels, industrial chemicals and other chemicals made from the transgenic plants can be carbon neutral as such fuels, industrial chemicals and other chemicals can be made largely from CO₂ fixed out of the atmosphere.

The transgenic plants described herein also can be used in many different food production applications. For example, transgenic plants with cholesterol oxidase in their chloroplasts can use light more efficiently and this can expand the latitudes at which a plant can grow. Global warming may shift the fertile regions to more Northern and Southern latitudes where the light intensity can be reduced. The transgenic plants described herein can thrive under these less optimal light conditions because of their more efficient use of light, thus allowing many crops to be efficiently grown at more Northernly and Southernly latitudes. The transgenic plants can also be used to extend the growing season, increase crop yield, reduce time for crops to reach maturity, increase root crop yield, and increase the starch in crops so less harvest provides the same amount of energy value. This can increase crop yields per hectare producing more food as well as removing more CO₂ from the atmosphere.

The engineering of cholesterol oxidase into the chloroplasts of the transgenic plants can improve the efficiency and starch accumulation in all crops. All these transgenic plants will fix more CO₂ from the atmosphere and produce more starch compared to the wild-type plants.

The transgenic plants can produce greater root biomass which can favorably alter soil quality. The increased root biomass also increase carbon sequestration in the soil reducing the percent of carbon in the air. Increased root biomass will increase surface area of the roots allowing greater association between roots and soil bacteria and/or fungi. If the root has greater size (surface area) the root will hold more soil and thus inhibit the erosion of the topsoil (soil sustainability). Increasing the amount of topsoil retained and improving the quality of the soil (more bacteria and more topsoil retained) can increase the amount of food that is produced (and increase food security). In addition, transgenic roots from the previous year's plants will stay in the soil and will provide more food (biomass) for the consumption by beneficial soil insects and beneficial soil bacteria and fungi (increasing the number of beneficial insects, bacteria, and fungi) increasing soil quality over time. Improved soil quality can increase the yield from crops in the next year, producing a virtuous cycle of soil improvement and higher crop yields. Soil Nitrogen will also be improved the second year and in subsequent years (more retained root biomass equals more Nitrogen in the soil). Larger roots can support larger plants as the amount of water absorbed is directionally proportional to the biomass accumulated in terrestrial higher plants.

In some embodiments, alfalfa, almonds, asparagus, avocado, banana, barley, bean, blackberry, brassicas, broccoli, cabbage, cannabis, canola, carrot, cauliflower, celery, cherry, chicory, citrus, coffee, cotton, cucumber, eucalyptus, hemp, lettuce, lentil, maize, mango, melon, oat, papaya, pea, peanut, pineapple, plum, potato (including sweet potatoes), pumpkin, radish, rapeseed, raspberry, rice, rye, sorghum, soybean, spinach, strawberry, sugar beet, sugarcane, sunflower, tobacco, tomato, turnip, wheat, zucchini, and other fruiting vegetables (e.g. tomatoes, pepper, chili, eggplant, cucumber, squash etc.), other bulb vegetables (e.g., garlic, onion, leek etc.), other pome fruit (e.g. apples, pears etc.), other stone fruit (e.g., peach, nectarine, apricot, pears, plums etc.), Arabidopsis species, woody plants such as coniferous and deciduous trees, an ornamental plant, a perennial grass, a forage crop, flowers, other vegetables, other fruits, other agricultural crops, herbs, grasses, or perennial plant parts (e.g., bulbs; tubers; roots; crowns; stems; stolons; tillers; shoots; cuttings, including un-rooted cuttings, rooted cuttings, and callus cuttings or callus-generated plantlets; apical meristems etc.) are transformed with cholesterol oxidase to increase starch accumulation, and/or to reduce the need of these plants for sunlight allowing the plants to be grown in poorer light (e.g., at higher latitudes). The cholesterol oxidase can be transformed into alfalfa, almonds, asparagus, avocado, banana, barley, bean, blackberry, brassicas, broccoli, cabbage, cannabis, canola, carrot, cauliflower, celery, cherry, chicory, citrus, coffee, cotton, cucumber, eucalyptus, hemp, lettuce, lentil, maize, mango, melon, oat, papaya, pea, peanut, pineapple, plum, potato (including sweet potatoes), pumpkin, radish, rapeseed, raspberry, rice, rye, sorghum, soybean, spinach, strawberry, sugar beet, sugarcane, sunflower, tobacco, tomato, turnip, wheat, zucchini, and other fruiting vegetables (e.g. tomatoes, pepper, chili, eggplant, cucumber, squash etc.), other bulb vegetables (e.g., garlic, onion, leek etc.), other pome fruit (e.g. apples, pears etc.), other stone fruit (e.g., peach, nectarine, apricot, pears, plums etc.), Arabidopsis species, woody plants such as coniferous and deciduous trees, an ornamental plant, a perennial grass, a forage crop, flowers, other vegetables, other fruits, other agricultural crops, herbs, grasses, or perennial plant parts (e.g., bulbs; tubers; roots; crowns; stems; stolons; tillers; shoots; cuttings, including un-rooted cuttings, rooted cuttings, and callus cuttings or callus-generated plantlets; apical meristems etc.)

In an aspect, C3 photosynthetic plants are transformed with cholesterol oxidase to increase starch accumulation and/or reduce the sunlight needs of the plants. Such C3 photosynthetic plants include, for example, alfalfa (lucerne), barley, broad bean, cassava, Chlorella, cotton, cowpea, Eucalyptus, green beans, oats, rye, wheat, peanuts, potatoes, rice, spinach, soybean, sugar beets, sunflower, tomatoes, and most trees. Still other C3 plants include, for example, lawn grasses such as fescue and Kentucky bluegrass, evergreen trees and shrubs of the tropics, subtropics, and the Mediterranean, temperate evergreen conifers like the Scotch pine (Pinus sylvestris), deciduous trees and shrubs of the temperate regions, e.g. European beech (Fagus sylvatica), as well as weedy plants like the water hyacinth (Eichornia crassipes), lambsquarters (Chenopodium album), bindweed (Convolvolus arvensis), and wild oat (Avena fatua).

In an aspect, the transgenic plants with increased starch accumulation can be used in fermentation. For such uses and related used, the plants may be subject to pretreatment. Conventional methods Include physical, chemical, and/or biological pretreaments. For example, physical pretreatment techniques can include one or more of various types of milling, crushing, irradiation, steaming/steam explosion, and hydrothermolysis. Chemical pretreatment techniques can include acid, alkaline, organic solvent, ammonia, sulfur dioxide, carbon dioxide, and pH-controlled hydrothermolysis. Biological pretreatment techniques can involve applying lignin-solubilizing microorganisms (T.-A. Hsu, “Handbook on Bioethanol. Production and Utilization”, C. E. Wyman (Ed.), 1996, Taylor & Francis: Washington, D.C., 179-212; P. Ghosh and A. Singh, A., Adv. Appl. Microbiol., 1993, 39: 295-333; J. D. McMillan, in “Enzymatic Conversion of Biomass for Fuels Production”, M. Himmel et al., (Eds.), 1994, Chapter 15, ACS Symposium Series 566, American Chemical Society; B. Hahn-Hagerdal, Enz. Microb. Tech., 1996, 18: 312-331; and L. Vallander and K. E. L. Eriksson, Adv. Biochem. Eng./Biotechnol., 1990, 42: 63-95). The purpose of the pretreatment step is to break down the lignin and carbohydrate structure to make the cellulose fraction accessible to cellulolytic enzymes.

The plant material may also be subject to saccharification. In saccharification (or enzymatic hydrolysis), lignocellulose is converted into fermentable sugars by lignocellulolytic enzymes present in the pretreated material or exogenously added. Saccharification is generally performed in stirred-tank reactors or fermentors under controlled pH, temperature, and mixing conditions. A saccharification step may last up to 200 hours. Saccharification may be carried out at temperatures from about 30 C. to about 65 C., in particular around 50 C., and at a pH in the range of between about 4 and about 5, in particular, around pH 4.5. Saccharification can be performed on the whole pretreated material.

In the fermentation step, sugars, released from the lignocellulose as a result of the pretreatment and enzymatic hydrolysis steps, are fermented to one or more organic substances, e.g., ethanol, by a fermenting microorganism, such as yeasts and/or bacteria. The fermentation can also be carried out simultaneously with the enzymatic hydrolysis in the same vessels, again under controlled pH, temperature and mixing conditions. When saccharification and fermentation are performed simultaneously in the same vessel, the process is generally termed simultaneous saccharification and fermentation or SSF.

Fermenting microorganisms and methods for their use in ethanol production are known in the art (Sheehan, “The road to Bioethanol: A strategic Perspective of the US Department of Energy's National Ethanol Program” In: “Glucosyl Hydrolases For Biomass Conversion”, ACS Symposium Series 769, 2001, American Chemical Society; Washington, D.C.). Existing ethanol production methods that utilize corn grain as the biomass typically involve the use of yeast, particularly strains of Saccharomyces cerevisiae. Such strains can be utilized in the methods of the invention. While such strains may be preferred for the production of ethanol from glucose that is derived from the degradation of cellulose and/or starch, the methods of the present invention do not depend on the use of a particular microorganism, or of a strain thereof, or of any particular combination of said microorganisms and said strains.

Microorganisms and engineered microorganisms that can utilize the transgenic plants as carbon sources to make biofuels, industrial chemicals, and other chemicals include, for example, the butanediol producing organism described in U.S. Application publication No. US20200095616, the butadiene producing organisms of US20200115722A1, US20200040366A1, the adipic acid producing organisms of US20200080064A1, the aliphatic alcohol or acid producing organisms of US20200056213A1, the ethylene glycol producing organisms of US20190185888A1, the glucose fermenting organisms of US20190017079A1, the organisms of US20180282827A1, the polymer, fuel or fuel additive producing organisms of US20210040012A1, the propanol, alcohol and polyol producing organisms of US20200325500A1, and the microalgae organisms of US20160122787A1 and US20150275149A1, all of which are incorporated by reference in their entirety for all purposes.

Yeast or other microorganisms are typically added to the hydrolysate and the fermentation is allowed to proceed for 24-96 hours, such as 35-60 hours. The temperature of fermentation is typically between 26-40 C, such as 32 C, and at a pH between 3 and 6, such as about pH 4-5.

A fermentation stimulator may be used to further improve the fermentation process, in particular, the performance of the fermenting microorganism, such as, rate enhancement and ethanol yield. Fermentation stimulators for growth include vitamins and minerals. Examples of vitamins include multivitamin, biotin, pantothenate, nicotinic acid, meso-inositol, thiamine, pyridoxine, para-aminobenzoic acid, folic acid, riboflavin, and vitamins A, B, C, D, and E (Alfenore et al., “Improving ethanol production and viability of Saccharomyces cerevisiae by a vitamin feeding strategy during fed-batch process”, 2002, Springer-Verlag). Examples of minerals include minerals and mineral salts that can supply nutrients comprising phosphate, potassium, manganese, sulfur, calcium, iron, zinc, magnesium and copper.

The transgenic plants and plant parts disclosed herein can be used in methods involving combined hydrolysis of starch and of cellulosic material for increased product yields (e.g., chemicals such as ethanol and other industrial useful chemicals). In addition to providing enhanced yields of products (e.g., ethanol), these methods can be performed in existing starch-based processing facilities.

Starch is a glucose polymer that is easily hydrolyzed to individual glucose molecules for fermentation. Starch hydrolysis may be performed in the presence of an amylolytic microorganism or enzymes such as amylase enzymes. Starch hydrolysis can be performed in the presence of at least one amylase enzyme. Examples of suitable amylase enzymes include alpha-amylase (which randomly cleaves the alpha(1-4)glycosidic linkages of amylose to yield dextrin, maltose or glucose molecules) and glucoamylase (which cleaves the α(1-4) and α(1-6)glycosidic linkages of amylose and amylopectin to yield glucose).

Hydrolysis of starch and hydrolysis of cellulosic material can be performed simultaneously (i.e., at the same time) under identical conditions (e.g., under conditions commonly used for starch hydrolysis). Alternatively, the hydrolytic reactions can be performed sequentially (e.g., hydrolysis of lignocellulose can be performed prior to hydrolysis of starch). When starch and cellulosic material are hydrolyzed simultaneously, the conditions are preferably selected to promote starch degradation and to activate lignocellulolytic enzyme(s) for the degradation of lignocellulose. Factors that can be varied to optimize such conditions include physical processing of the plants or plant parts, and reaction conditions such as pH, temperature, viscosity, processing times, and addition of amylase enzymes for starch hydrolysis.

The methods may use transgenic plants (or plant parts) alone or a mixture of non-transgenic plants (or plant parts) and plants (or plant parts) transformed according to the present invention. Suitable plants include any plants that can be employed in starch-based ethanol production (e.g., corn, wheat, potato, cassaya, etc). For example, the present inventive methods may be used to increase ethanol yields from corn grains.

The transgenic plants can find use in biomass conversion methods for producing sugars or biofuels from plant biomass. Herein, the term “biofuels” refers to any fuel derived from harvested plant parts. Biofuels comprise but are not limited to biodiesel, vegetable oils, bioalcohols (i.e. ethanol, methanol, propanol, butanol, etc.) and biogases (i.e. methane). The transgenic plants can be engineered to accumulate higher concentrations of starch in their green tissues thus providing a rich source of carbohydrates which then can be converted to biofuels. Herein, the term “free sugars” defines any carbohydrate derived from plant biomass that can be further processed to make fermentable sugars, chemicals, biofuels, plastics, feed additives or any other commercially important product. In an aspect, plant biomass can be engineered to down-regulate the activity of one or more starch degradation enzymes. The resultant plant will contain increased levels of starch which then can be converted to free sugars in a conventional biomass conversion method. Herein, the term “biomass conversion method” defines any process that converts plant parts into fermentable sugars, biofuels, chemicals, plastics, feed additives, or any other commercially important products. Biomass conversion methods may also contain a subcategory herein referred to as a “non-animal feed biomass conversion method”. Non-animal feed biomass conversion method defines any process that converts plant parts into fermentable sugars, biofuels, chemicals and plastics not destined for animal consumption.

The transgenic plants described herein are useful in the production of dextrose for fructose syrups, specialty sugars, and in alcohol and other end-product (e.g. organic acid, ascorbic acid, and amino acids) production from fermentation of starch (G. M. A van Beynum et al., Eds. (1985) Starch Conversion Technology, Marcel Dekker Inc. NY). Production of alcohol from the fermentation of starch derived from the green tissues of the plants of the invention may include the production of fuel alcohol or potable alcohol. The alcohol can be ethanol. In particular, alcohol fermentation production processes are characterized as wet milling or dry milling processes. In some embodiments, the plants are subjected to a wet milling fermentation process and, in other embodiments, a dry milling process is used. In certain embodiments, ethanol may be produced using a raw starch hydrolysis method.

Dry grain milling involves a number of basic steps, which generally include: grinding, cooking, liquefaction, saccharification, fermentation and separation of liquid and solids to produce alcohol and other co-products. Plant material and particularly whole cereal grains, such as maize, wheat or rye are ground. In some cases the grain may be first fractionated into component parts. The ground plant material may be milled to obtain a coarse or fine particle. The ground plant material is mixed with liquid in a slurry tank. The slurry is subjected to high temperatures in a jet cooker along with liquefying enzymes (e.g. alpha amylases) to solubilize and hydrolyze the starch in the cereal to dextrins. The mixture is cooled down and further treated with saccharifying enzymes to produce glucose. The mash containing glucose is then fermented for approximately 24 to 120 hours in the presence of fermentation microorganisms, such as ethanol producing microorganism and particularly yeast (Saccharomyces spp). The solids in the mash are separated from the liquid phase and alcohol such as ethanol and useful co-products such as distillers' grains are obtained.

The saccharification step and fermentation step can be combined and the process may be referred to as simultaneous saccharification and fermentation or simultaneous saccharification, yeast propagation and fermentation.

In other embodiments, the cooking step or exposure of the green starch containing substrate to temperatures above the gelatinization temperate of the starch in the substrate may be eliminated. These fermentation processes in some embodiments include milling of a cereal grain or fractionated grain and combining the ground cereal grain with liquid to form a slurry, which is then mixed in a single vessel with amylases, glucoamylases, and/or other enzymes having granular starch hydrolyzing activity and yeast to produce ethanol and other co-products (U.S. Pat. No.

4,514,496, WO 04/081193 and WO 04/080923). In some embodiments, the enzymes useful for fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme and other glucoamylases.

The transgenic plant can be a transgenic sugarcane containing high amounts of starch in its' green tissues. A sugarcane plant containing high starch may be desirable in conventional operations that employ cane sugar in a fermentation-distillation operation which may also utilize a high starch bagasse by-product as a high valued fuel source.

It may be beneficial to create a plant with increased green starch that has been further modified to express a processing enzyme that when activated will be capable of self-processing the substrate upon which it acts to obtain the desired result as described in, US20030135885 and U.S. Pat. No. 7,102,057 herein incorporated by reference. In accordance with the present invention, a “self-processing” plant or plant part has incorporated therein an isolated polynucleotide encoding a processing enzyme capable of processing, e.g., modifying, starches, polysaccharides, lipids, proteins, and the like in plants, wherein the processing enzyme can be mesophilic, thermophilic or hyperthermophilic, and may be activated by grinding, addition of water, heating, or otherwise providing favorable conditions for function of the enzyme. The isolated polynucleotide encoding the processing enzyme is integrated into a plant or plant part for expression therein. Upon expression and activation of the processing enzyme, the plant or plant part of the present invention processes the substrate upon which the processing enzyme acts. Therefore, the plant or plant parts of the present invention are capable of self-processing the substrate of the enzyme upon activation of the processing enzyme contained therein in the absence of or with reduced external sources normally required for processing these substrates. As such, the transformed plants, transformed plant cells, and transformed plant parts have “built-in” processing capabilities to process desired substrates via the enzymes incorporated therein according to this invention. Preferably, the processing enzyme-encoding polynucleotide are “genetically stable,” i.e., the polynucleotide is stably maintained in the transformed plant or plant parts of the present invention and stably inherited by progeny through successive generations.

Such self-processing plants and plant parts can eliminate the need to mill or otherwise physically disrupt the integrity of plant parts prior to recovery of starch-derived products. For example, improved methods for processing maize and other grains to recover starch-derived products can benefit from self-processing plants. Methods useful herein can also allow the recovery of starch granules that contain levels of starch degrading enzymes, in or on the granules that are adequate for the hydrolysis of specific bonds within the starch without the requirement for adding exogenously produced starch hydrolyzing enzymes.

In addition, the “self-processing” transformed plant part, e.g., grain, and transformed plant avoid major problems with existing technology, i.e., processing enzymes are typically produced by fermentation of microbes, which requires isolating the enzymes from the culture supernatants, which costs money; the isolated enzyme needs to be formulated for the particular application, and processes and machinery for adding, mixing and reacting the enzyme with its substrate must be developed. The transformed plant of the invention or a part thereof is also a source of the processing enzyme itself as well as substrates and products of that enzyme, such as sugars, amino acids, fatty acids and starch and non-starch polysaccharides. The plant of the invention may also be employed to prepare progeny plants such as hybrids and inbreds.

The inventions disclosed herein will be better understood from the experimental details which follow. However, one skilled in the art will readily appreciate that the specific methods and results discussed are merely illustrative of the inventions as described more fully in the claims which follow thereafter.

### EXAMPLES

### Example 1. Starch Accumulation in Leaves #2

Transgenic plants have been engineered to express cholesterol oxidase so that the membranes in the chloroplasts contain new sterols. Wild-type and transgenic plants tobacco plants were used. Leaves were sampled and analyzed for starch according to the method of Smith et al., Nature Protocols 1:1342-1345 (2006).

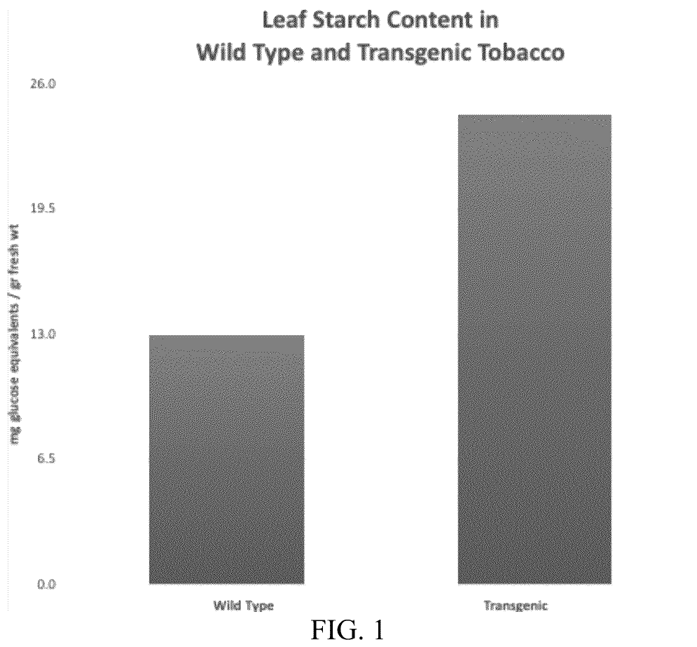

In FIG. 1 Starch was expressed as mg glucose equivalents per gram fresh weight of leaf. FIG. 1 shows that leaves from transgenic plants contain roughly two-fold higher levels of starch per gram of leaf fresh weight compared to wild-type leaves. Average leaf starch content from 3 wild type and 15 transgenic plants. Wild Type leaves averaged 12.9 (S.E. 4.3) mg glucose equivalents per gram fresh weight while Transgenic leaves averaged 24.4 (S.E. 2.7) mg glucose equivalents per gram fresh weight, a ratio of 1.9:1, Transgenic to Wild Type.

### Example 2. Photosynthesis in Transgenic Plants

Wild-type and transgenic tobacco plants as described in Example 1 can be used. Class C chloroplasts are isolated from the leaves of the plants. WCET is measured as uncoupled, methyl viologen dependent oxygen uptake in water-jacketed oxygen polarograph chambers with an oxygen electrode (YSI). Red filtered (>600 nm) actinic light is used to illuminate thylakoid membranes isolated from transgenic and control plants. Neutral density filters are used to alter the relative incident light on the reaction vessel. Measurements are made in the water-jacketed vessels and can be performed at different temperature such as 10, 25 and 35° C.

The chloroplasts, once thawed, lose activity over time, even when maintained on ice and in the dark. A best fit binomial equation for the decay in activity can be used to adjust each experimental measurement for the length of time the chloroplast sample had been thawed before the measurement is taken. WCET activity is plotted as a function of relative light intensity used in the measurements.

Transgenic thylakoid membranes exhibit 2-3-fold higher light use efficiencies than the control thylakoid membranes. The altered steroid composition of the thylakoid membranes in transgenic plants may result in two separate effects on photosynthetic electron transport, both which enhance photosynthetic capacity in the transgenic plants. Under light limiting conditions, the transgenic plant thylakoid membranes can have about a 2-fold improvement in light use efficiency, resulting in higher rates of electron transport than in control plant thylakoid membranes at the same light intensity. This could confer an advantage to transgenic plants grown in sub-optimal light conditions, e.g., in shade or with light of lower intensity (e.g., at a higher latitude). When light is not limiting, the transgenic thylakoid membranes can also exhibit about a 2-fold higher rate of WCET capacity.

### Example 3. Growth of Plants Under Light Limiting Conditions

Wild-type and transgenic tobacco plants as described in Example 1 were used. The plants were grown indoors with about 20% of outdoor light intensity. Light was provided by LED light sources. The plants were measured and compared for biomass, seed production, root mass, and development rate.

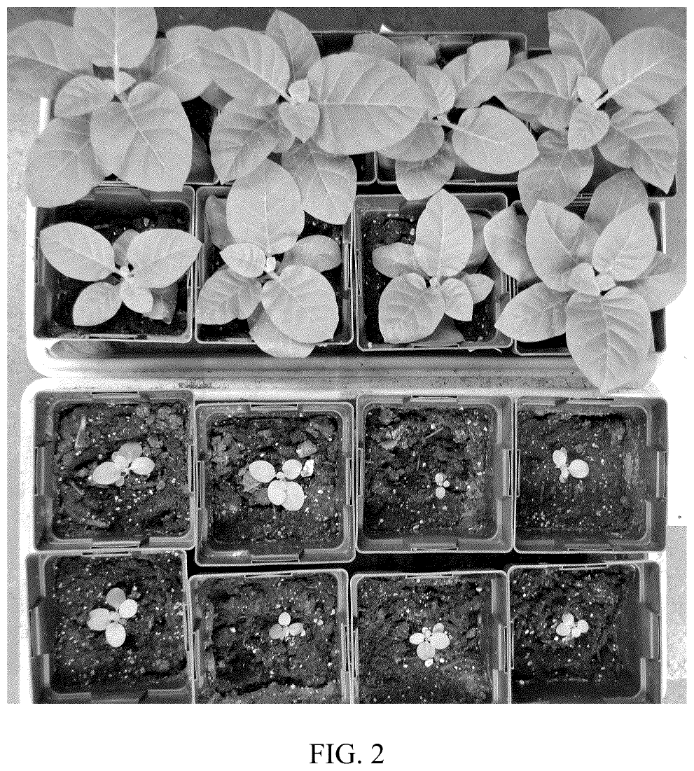

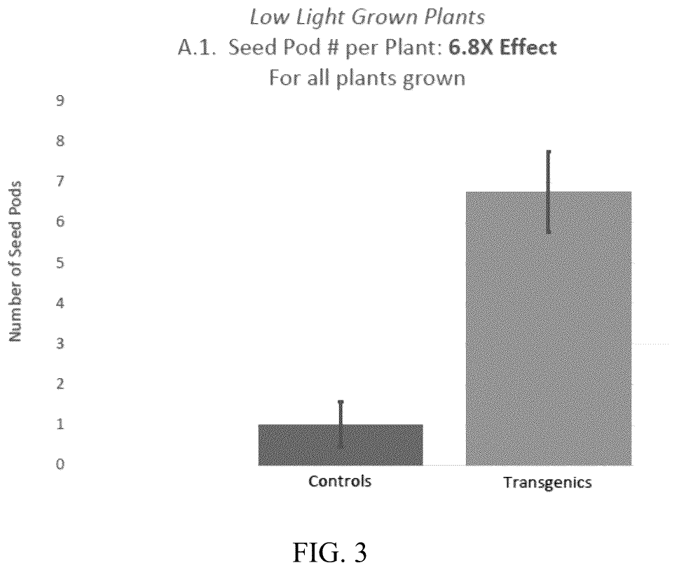

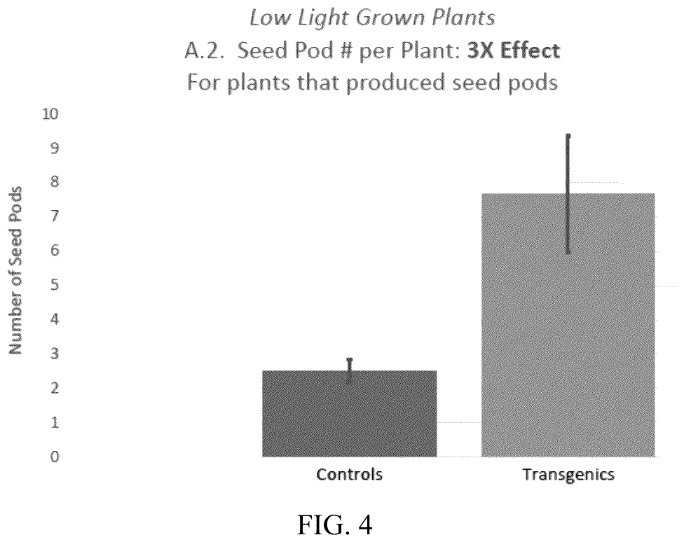

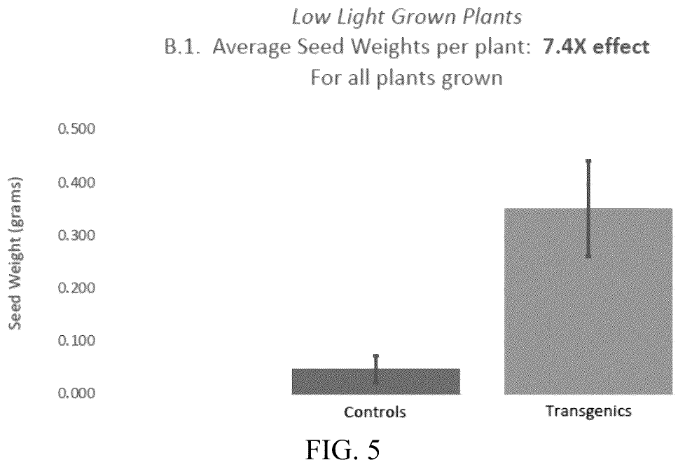

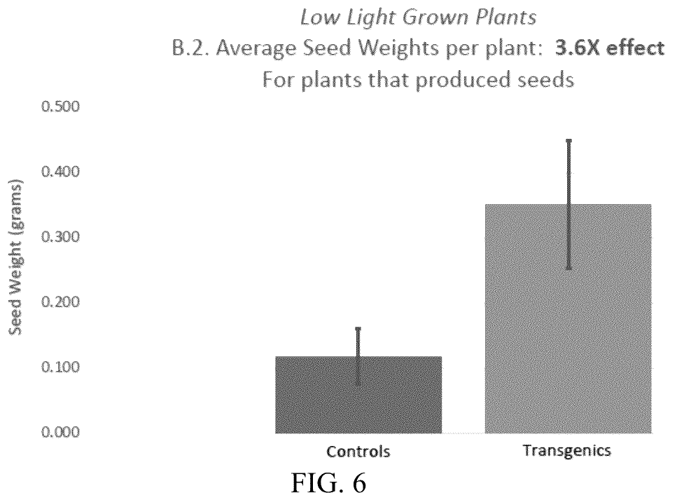

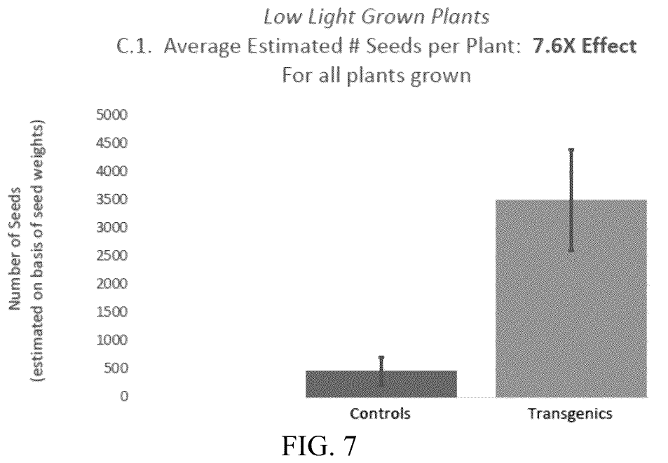

The comparative results for the transgenic and wild-type plants are shown in FIGS. 2-12. Transgenic plants grown at low light intensity exhibited dramatically elevated rates of photosynthetic activity, including photosynthetic light use efficiency. For photosynthetic light use efficiency, the enhancements were about the same as in full sunlight grown plants (about 2-fold), but the light-saturated rates of electron transport capacity were even greater: nearly 5-fold higher than the rates in chloroplasts from control plants. This compares to a 2-fold increase in transgenic plants grown in full sunlight.

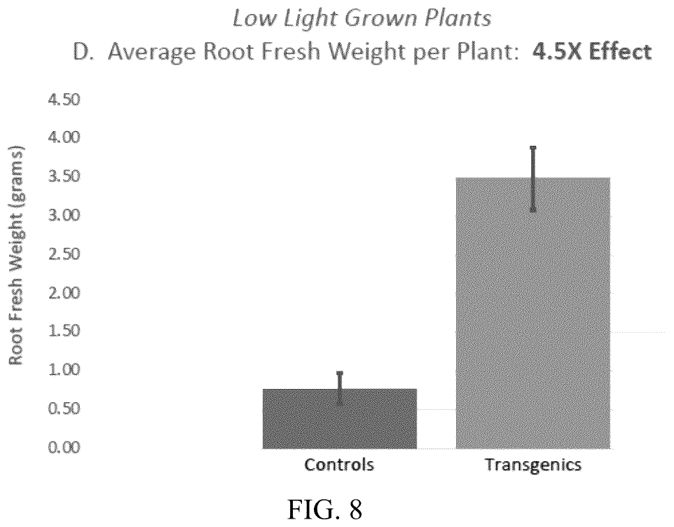

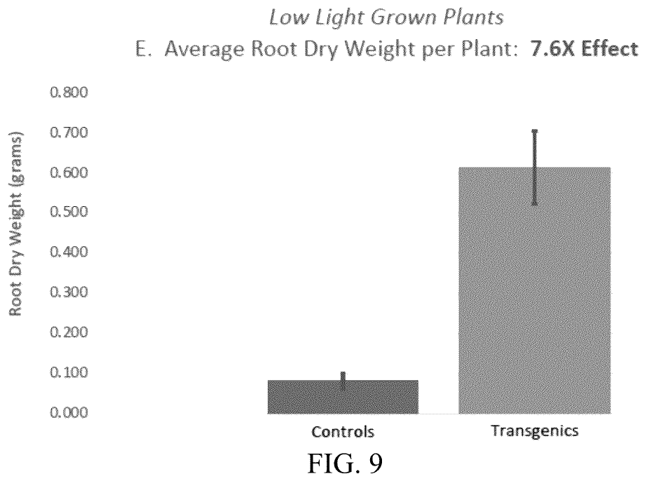

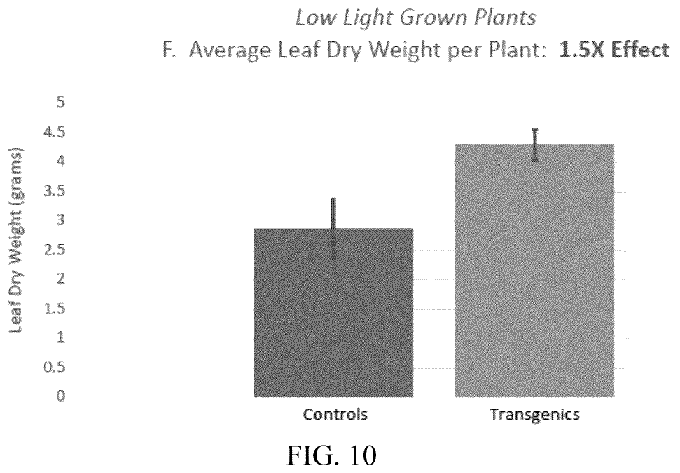

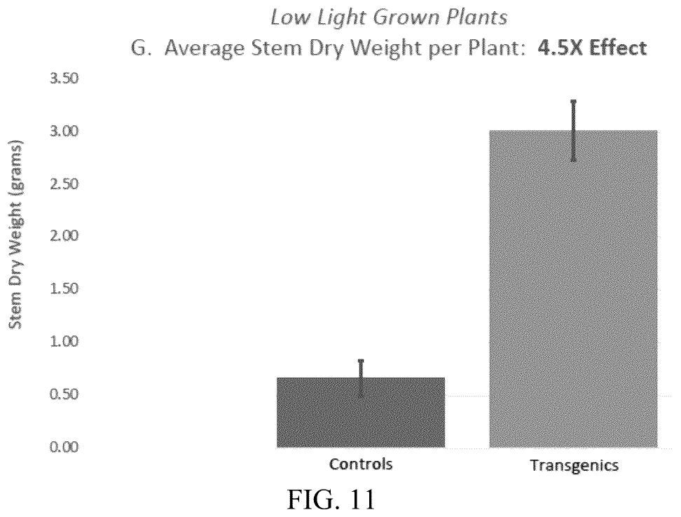

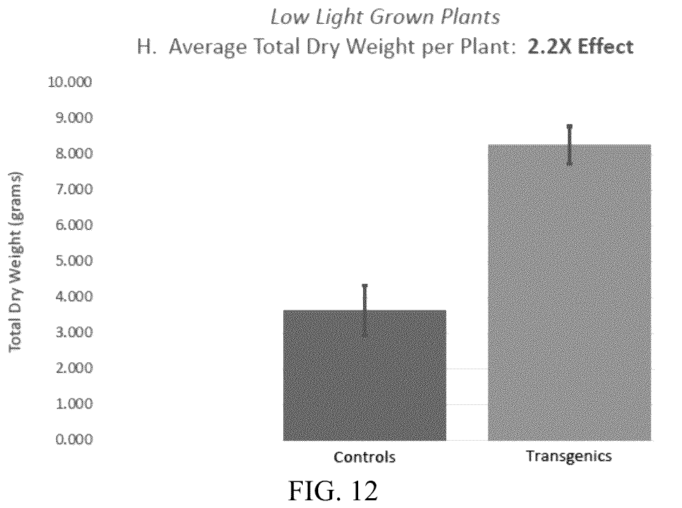

FIG. 2 shows pictures of the increase in growth of the transgenic plants compared to wild-type plants after about 7.5 weeks of growth under low light conditions. The eight large plants are transgenic and the eight small plants are wild-type. The transgenic plants had 4.5-fold greater root fresh weights (FIG. 8), 7.6-fold greater root dry biomass (FIG. 9), 1.5-fold greater leaf dry biomass (FIG. 10), 4.5-fold greater stem dry biomass (FIG. 11), 2.2-fold greater total dry biomass (FIGS. 12), 3.5- to 7.5-fold greater reproductive output. The transgenic plants reached flowering state in half the time as the control plants, and the percentage of plants flowering was 2-fold higher in the transgenic.

### Transgenic Plant Enhancements:

| | |
|---|---|
| Root Biomass | 7.5X |
| Seed Biomass | 3.6-7.4X |
| Stems Biomass | 4.5X |
| Leave Biomass | 1.5X |
| Total Biomass | 2.3X |
| Reproductive Output | 3.7-7.6X |
| Time to Flowering | 2X as fast |

The increased growth rate and reduced time to flowering shows that the transgenic plants mature at twice the speed of a wild-type plant, and could grow through two-crop cycles in the time it takes the wild-type to grow through one cycle.

### Example 4. Growth of Plants Under Sunlight

Wild-type and transgenic tobacco plants as described in Example 1 were used. The plants were grown outdoors on the roof of a building in Buffalo, New York from Jun. 1, 2021 to Sep. 15, 2021. The plants were measured and compared for biomass, seed production, root mass, and development rate.

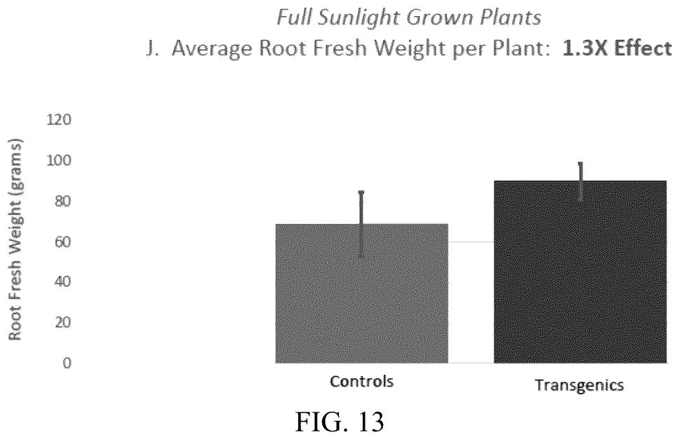

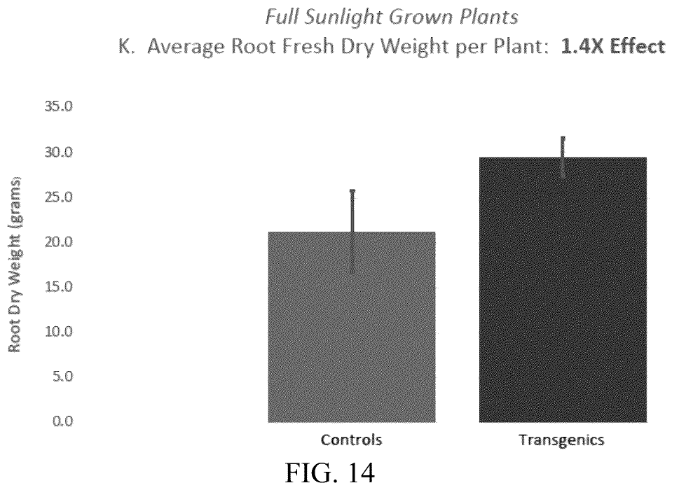

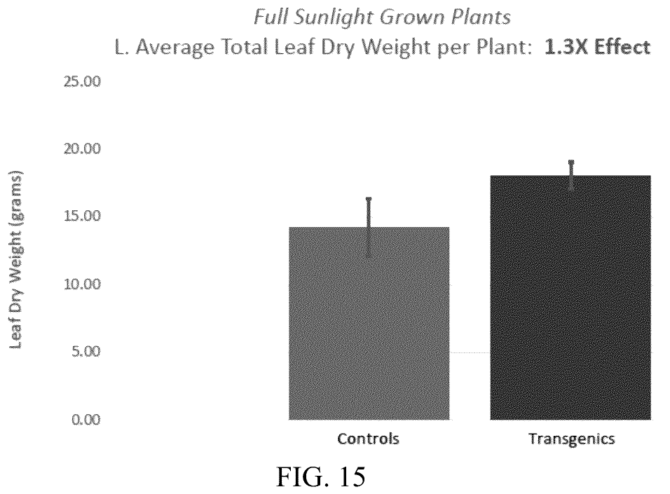

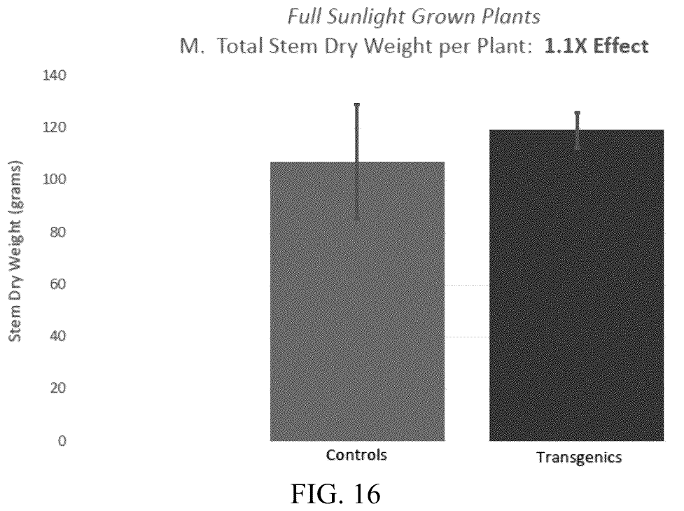

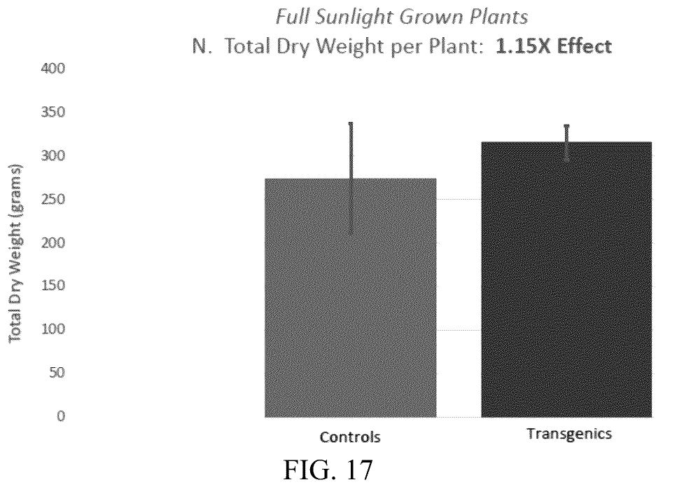

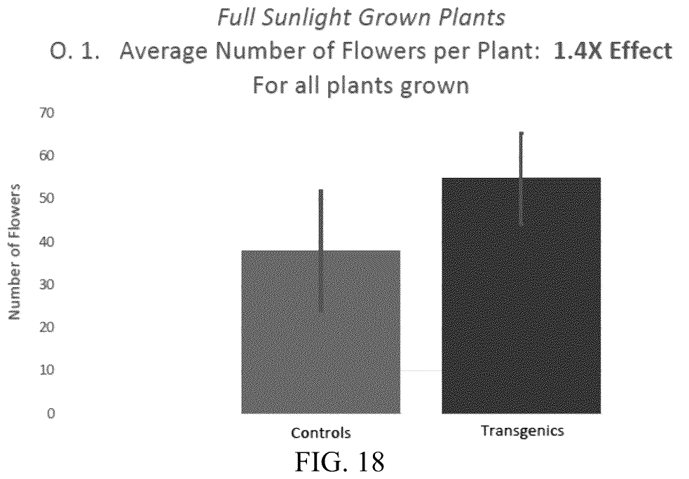

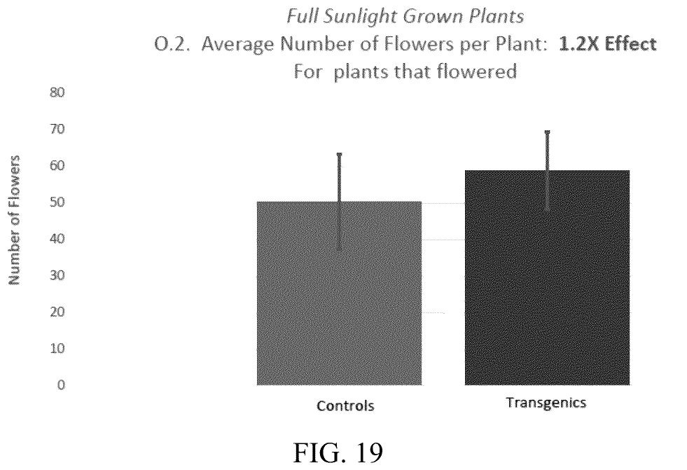

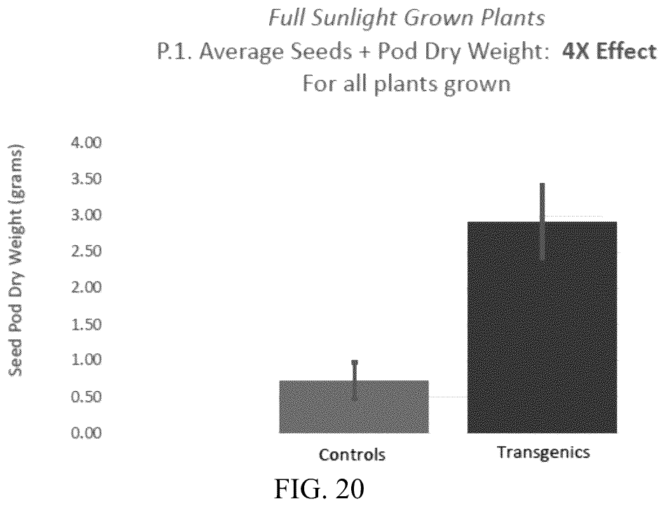

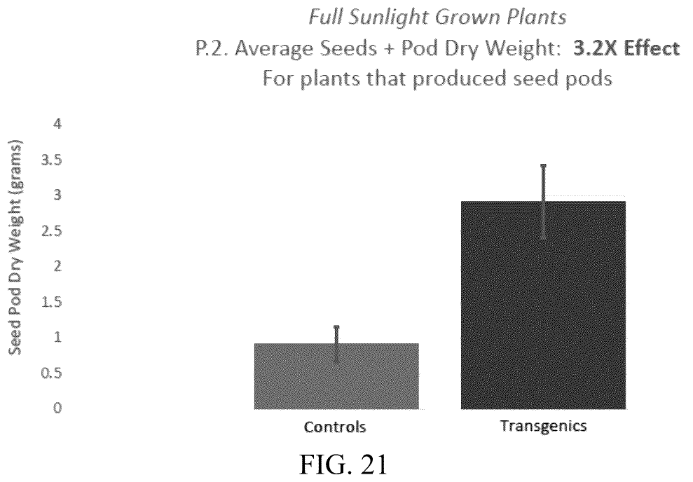

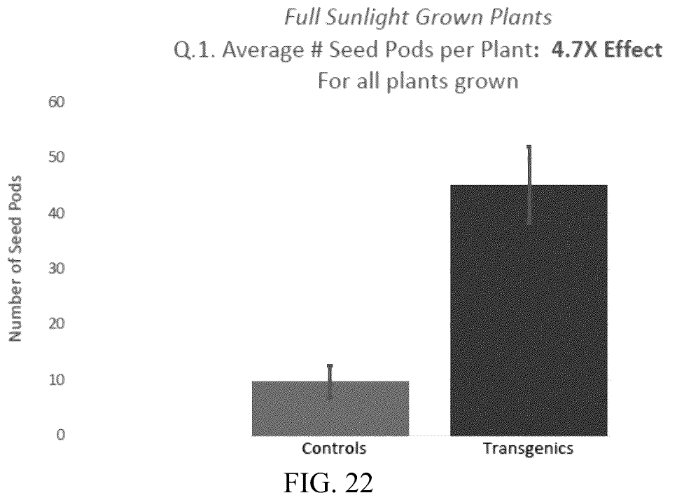

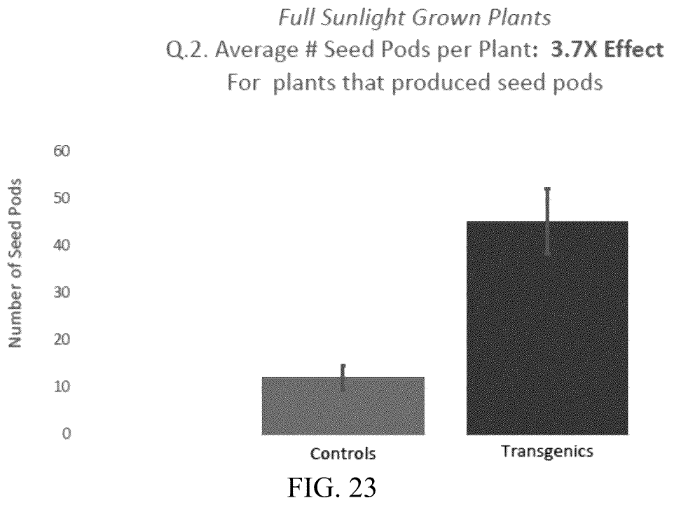

The comparative results for the transgenic and wild-type plants are shown in FIGS. 13-23. Grown at full sunlight, transgenic plants had 1.3-fold increase in root fresh weight (FIG. 13), 1.4-fold greater root dry biomass (FIG. 14), 1.3-fold greater leaf dry biomass (FIG. 15), 1.1-fold greater stem dry biomass (FIG. 16), 1.15-fold greater total dry biomass (FIG. 17), 1.2-1.4-fold greater number of flowers (FIGS. 18-19), 3.2- to 4-fold greater seeds+pod dry weight (FIGS. 20-21), 3.7- to 4.7-fold greater number of seed pods (FIGS. 22-23), and they reached flowering state in half the time as the control plants.

### Transgenic Plant Enhancements:

| | |
|---|---|
| Root Biomass | 1.4X |
| Stem Biomass | 1.1X |
| Leave Biomass | 1.3X |
| Total Biomass | 1.3X |
| Reproductive Output | 4.5X |
| Time to Flowering | 2X as fast |

The increased growth rate and reduced time to flowering shows that the transgenic plants mature at twice the speed of a wild-type plant, and could grow through two-crop cycles in the time it takes the wild-type to grow through one cycle.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. A method for increasing production in a crop plant, comprising the steps of: obtaining a crop plant wherein a chloroplast of the crop plant has a cholesterol oxidase; and growing the crop plant under suitable conditions, wherein the crop plant increases production of a starch, a root biomass, a leaf biomass, a number of flowers, a number of seeds, or a total biomass compared to a wild-type crop plant.

2. The method of claim 1, wherein the suitable conditions includes growing the crop plant at a latitude that would not be suitable for a wild-type crop plant.

3. The method of claim 1, wherein the crop plant is grown under a light limiting condition.

4. The method of claim 1, wherein the crop plant reaches a flowering twice as fast as a wild-type plant.

5. The method of claim 1, further comprising the step of growing the crop plant for a plurality of crop cycles.

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. The method of claim 1, wherein the crop plant reaches a flowering twice as fast as a wild-type plant.

21. (canceled)

22. The method of claim 1, wherein the cholesterol oxidase is from an actinomycetes.

23. The method of claim 1, wherein the cholesterol oxidase is from a Streptomyces sp. Strain A19249.

24. The method of claim 1, wherein the cholesterol oxidase has the sequence of GenBank Accession No. A19124.

25. The method of claim 1, wherein a root of the crop plant is increased in a size and an amount; and further comprising the step of leaving the roots in the soil after growth of the crop plant is finished.

26. The method of claim 1, further comprising the step of growing the crop plant under poor light conditions, wherein the crop plant grows better than the wild-type plant and produces about two-fold more starch compared to a wild-type crop plant.

27. The method of claim 26, wherein the suitable conditions includes growing the crop plant at a latitude that would not be suitable for a wild-type crop plant.

28. The method of claim 1, wherein the crop plant reaches maturity in half the time of a wild-type crop plant.

29. The method of claim 3, wherein the yield of the crop plant is increased over a wild-type crop plant grown under the same light limiting conditions.

30. The method of claim 1, wherein the crop plant increases CO₂ fixation about two-fold compared to the wild-type plant.

31. The method of claim 30, wherein the plant has a plurality of large leaves.

32. A method for increasing a CO2 fixation from the atmosphere, comprising the steps of: obtaining a microalgae wherein a chloroplast of the microalgae has a cholesterol oxidase; and growing the microalgae under suitable conditions, wherein the microalgae increases CO2 fixation about two-fold compared to the wild-type microalgae.

33. The method of claim 32, wherein the microalgae has been engineered to have a biosynthetic pathway for making the biofuel.

34. The method of claim 32, wherein the microalgae has been engineered to have a biosynthetic pathway for making an industrial chemical, wherein the amount of the industrial chemical made is about twice that made from a wild-type microalgae with the biosynthetic pathway for making the industrial chemical.

35. The method of claim 34, wherein the industrial chemical is a butanediol.
